SEQUENCE LISTING (2 sequences)

SEQ ID NO: 1 - DNA, 19 bp, Homo sapiens
ggtgtgcagttggaatgta

SEQ ID NO: 2 - DNA, 19 bp, Homo sapiens
gaggcagagtggactattt

(19) United States
(12) Patent Application Publication
(10) Pub. No.: US 2011/0053804 A1
(43) Pub. Date: Mar. 3, 2011

(54) Gene Signatures for the Prognosis of Cancer

(75) Inventors: Joan Massague, New York, NY (US); Xiang Zhang, New York, NY (US); David Padua, New York, NY (US)

(73) Assignee: SLOAN-KETTERING INSTITUTE FOR CANCER RESEARCH, New York, NY (US)

(21) Appl. No.: 12/935,224

(22) PCT Filed: Apr. 3, 2009
(86) PCT No.: PCT/US09/39469

§ 371 Date: Sep. 28, 2010

Related U.S. Application Data
(60) Provisional application No. 61/072,851, filed on Apr. 3, 2008.

(51) Int. Cl. C12Q 1/68; C40B 40/08; C12N 5/09

(52) U.S. Cl. 506/17; 435/6; 435/375

(57) ABSTRACT

The cytokine TGFβ, in the tumor microenvironment, primes cancer cells for metastasis to the lungs. TGFβ response status (TBRS) can be determined by comparing expression levels of a panel of genes from cancer cells to the expression levels of the same genes in epithelial cell lines before and after induction with TGFβ. A TGFβ gene response signature reveals a clinical association between TGFβ activity in primary estrogen receptor negative (ER−) tumors and risk of lung metastasis. Further, combining the gene signature of the present invention with the known lung metastasis signature (LMS) increases the predictive value of the LMS considerably.

This application claims the benefit of the filing date of U.S. Provisional Application No. 61/072,851, filed on Apr. 3, 2008, the disclosure of which is incorporated herein by reference.

### FIELD OF THE INVENTION

The present invention provides a gene expression signature useful to predict the risk of lung metastases in a breast cancer patient. Moreover, the signature of the present invention is useful to predict the time duration of lung metastasis free survival in a cancer patient. Further, this invention can predict the likelihood of responsiveness to anti-transforming growth factor beta (TGFβ) pathway therapy of a specific cancer tumor.

### BACKGROUND OF THE INVENTION

Metastasis refers to the spread of cancerous cells from the site of the primary tumor to non-contiguous organs. The presence or absence of metastasis often determines treatment as well as survival. The prediction of metastatic potential is thus an important component of cancer management. The overexpression or underexpression of certain genes has been shown to be related to the propensity of tumors to metastasize to the lungs (Minn et al., 2005) or bones (Kang et al., 2003b; Lynch et al., 2005; Yin et al., 1999). In some cases, the overexpression of certain genes has been linked to the production of, or responsiveness to, certain mediators that can actually influence the tumor cells and confer on them the ability to seed other organs and survive there. The microenvironment of the tumor, including the presence of cytokines, growth factors and proteases, could influence the ability of tumor cells to metastasize (Sleeman et al., 2007, McSherry et al., 2007). The cytokine TGFβ that has been implicated in the modulation of tumor progression in various experimental systems (Akhurst and Derynck, 2001; Bierie and Moses, 2006; Dumont and Arteaga, 2003; Siegel and Massagué, 2003; Wakefield and Roberts, 2002). Low expression levels of TGFβ receptors in ER− tumors is associated with better overall outcome (Buck et al., 2004), whereas overexpression of TGFβ is associated with a high incidence of distant metastasis (Dalal et al., 1993).

A previously described gene signature has been shown to be predictive of lung metastasis of cancer. The LMS is a set of 17 genes (Table 1) whose expression in ER− tumors indicates a high risk of pulmonary relapse in patients (Minn et al., 2007). Several of these genes have been validated as mediators of lung metastasis (Gupta et al., 2007a; Gupta et al., 2007b; Gupta, 2007; Minn et al., 2005).

### SUMMARY OF THE INVENTION

The cytokine TGFβ, in the tumor microenvironment, primes cancer cells for metastasis to the lungs. A TGFβ gene response signature reveals a clinical association between TGFβ activity in primary estrogen receptor negative (ER−) tumors and risk of lung metastasis. Further, combining the gene signature of the present invention with the known lung metastasis signature (LMS) increases the predictive value of the LMS considerably.

TGFβ response status (TBRS) can be determined by comparing expression levels of a panel of genes from cancer cells to the expression levels of the same genes in epithelial cell lines before and after induction with TGFβ. While a total of 153 genes were found to be involved in the response, smaller subsets of these genes may be used to determine the signature.

The TBRS provides a method of diagnosing metastatic potential of cancer comprising obtaining a diagnostic signature from cancer cells indicative of the metastatic potential of the cancer cells, wherein this diagnostic signature is obtained by measuring levels in cancer cells from the patient of five or more markers selected from the group of genes typifying the TGFβ response in human epithelial cells. This diagnostic signature is compared to a control signature; and based on the comparison, a prognosis of a high risk for metastasis is given if the diagnostic signature is different from the control signature by at least a threshold amount.

This method may be used in melanoma, breast cancer, colon carcinoma, and other types of cancer.

### BRIEF DESCRIPTION OF THE FIGURES

FIGS. 1A-C. (A) and (B) Kaplan-Meier curves representing the probability of cumulative lung (left panel) or bone (right panel) metastasis-free survival for this cohort. Tumors are categorized according to their TBRS and ER status. (C) Lung metastasis-free survival restricted to patients with ER− negative tumors. Patients were categorized according to their TBRS and LMS status.

FIGS. 2A-E. (A) Schematic of lung metastasis assay from an orthotopic breast cancer inoculation. (B) Immunoblots using indicated antibodies were performed on whole-cell extracts from control, Smad4 knockdown, and Smad4-Rescue LM2 cells. (C) Mice injected with 5×10⁵ cells into the fourth mammary fat pads were measured for tumor size at day 28. (D) Blood from tumor-bearing mice was isolated and red blood cells lysed. RNA from the remaining cells was extracted for qRT-PCR. The presence of circulating tumor cells was assessed as a function of human-specific GAPDH expression relative to murine β2-microglobulin, in 3 mL of mouse blood perfusate. (E) Bioluminescent quantification of lung metastases elicited from orthotopically implanted breast tumors. Orthotopic tumors were grown to approximately 300 mm³, mastectomies were performed and lung metastases were quantified using bioluminescence imaging seven days later.

FIGS. 3A-D. (A) LM2 cells and a clinically-derived pleural effusion sample (CN34.2A) were pretreated with TGFβ for 6 h. LM2 (2×10⁵) and CN34.2A (4×10⁵) cells were injected into the lateral tail vein and lung colonization was analyzed by in vivo bioluminescence imaging. (B) Bar graph represents 24 h time point measurements of the normalized photon flux from animals injected with either LM2 or CN34.2A cells. (C) Bone metastasis assays were performed by intracardiac injection of LM2 or BoM-1833 cells (3×10⁴). Samples were pretreated with 100 μM of TGFβ for 6 h and compared to an untreated control. (D) Bar graphs represent seven-day time point analysis of the normalized photon flux from the mouse hind limbs.

FIGS. 4A-C. (A) Microarray and qRT-PCR analysis for the four epithelial cell lines treated with TGFβ. Fold change values for the TGFβ induction of ANGPTL4 are indicated. (B) Box-and-whisker plot comparing ANGPTL4 and NEDD9 TBRS-negative and -positive ER-negative tumors from the MSK/EMC cohorts. (C) TGFβ-induced changes in the mRNA expression of LMS genes in a panel of clinically derived pleural effusion samples and LM2 cells. Cells were treated with 100 μM of TGFβ for 3 h and analyzed by qRT-PCR using primers for the indicated genes. ER status for each is shown.

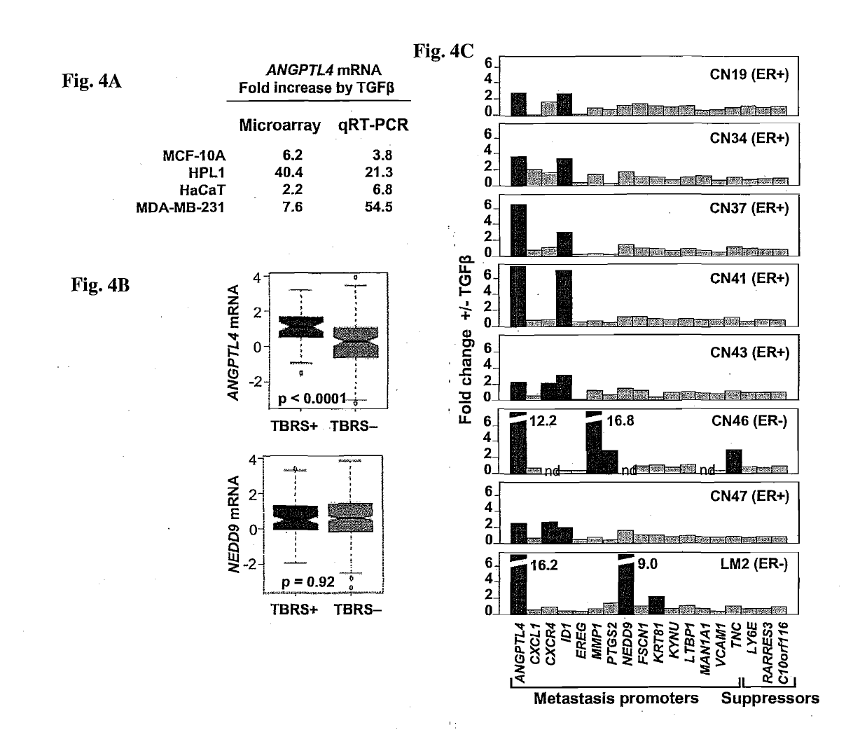

FIGS. 5A-J. Box-and-whisker plots comparing the RNA expression of TGFβ1, TGFβ2, TGFβ3, and LTBP1 TGFβR1, TGFβR2, TGFβR3, SMAD2, SMAD3 and SMAD4 in TBRS positive or negative primary breast tumors.

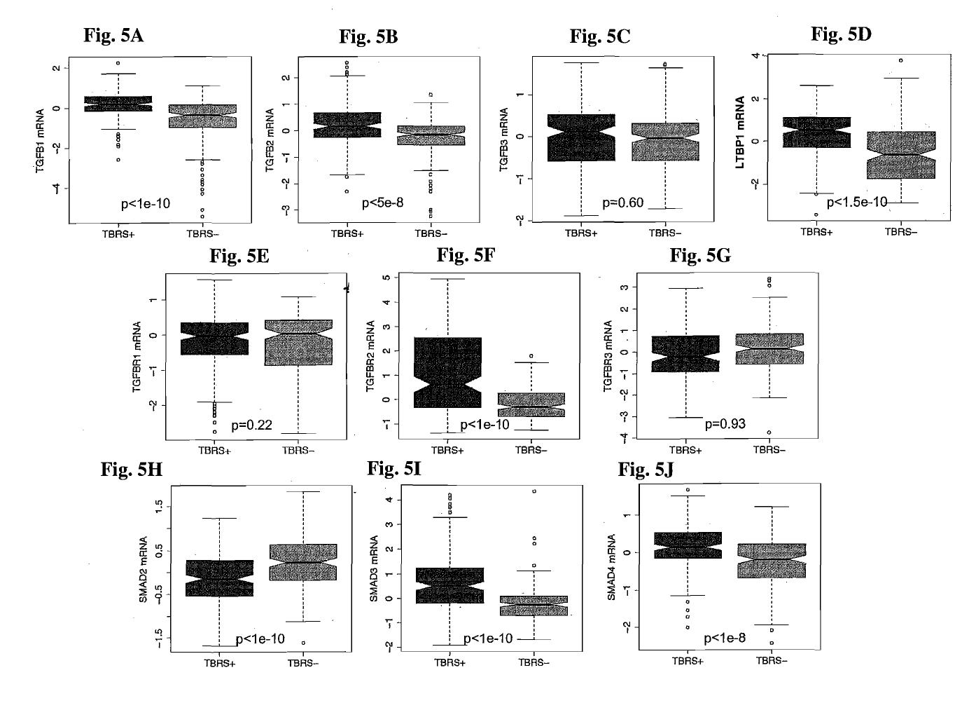

FIGS. 6A-B Kaplan-Meier curves of brain and liver metastasis-free survival from ER negative breast cancer patients either TBRS positive or negative.

FIGS. 7A-D Kaplan-Meier curves of ER negative breast cancer patients comparing TBRS status and other markers of poor clinical outcome such as large size, poor-prognosis signature positive, wound signature positive, and basal molecular subtype tumor designation.

FIGS. 8A-D Kaplan-Meier curves of breast cancer patients comparing LMS status and other markers of poor clinical outcome such as large size, poor-prognosis signature positive, wound signature positive, and basal molecular subtype designation.

FIGS. 9A-C Analysis of FIG. 1 data using ER status determined by microarray probe levels rather than using clinical pathology designations.

FIG. 10 Kaplan-Meier curves of melanoma patients comparing 153 gene TBRS signatures.

FIGS. 11A-B Kaplan-Meier curves of melanoma patients comparing 20 gene TBRS signatures.

### DESCRIPTION OF THE INVENTION

The present invention is based on the identification of a set of genes that can predict the risk of lung metastases developing in a cancer patient.

### Method for Diagnosing Metastatic Potential of Cancer Cells

One embodiment of the present invention is a method of determining the TGFβresponse status (TBRS) in cancer cells from a cancer patient. In another embodiment of the present invention, expression levels of all the 153 genes listed in Table 2 (TBRS153) are evaluated in a sample of cancer cells from a cancer patient using Affymetrix chips and these expression levels are, then compared, using statistical analysis, to the expression levels of the same genes in four epithelial cell lines before and after induction with TGFβ. According to the results of this comparison, the tumor of the patient is classified as either TBRS+ or TBRS− based on whether markers are at a higher or lower level than a threshold level. If the tumor is classified as TBRS+ and the tumor is further determined to be estrogen receptor negative, the cancer patient is determined to have a higher risk of lung metastasis than if the tumor was classified as TBRS−. This TBRS or signature can be determined with a combination of the 153 genes, in addition to looking at additional genes.

It will be appreciated that the determination of specific numerical values for the threshold is dependent on the particular tests that are included in the formation of the signatures, and the level of risk that is to be assigned as high risk. By way of example, however, when the markers are tested using the procedures described below, and the sum of the expression levels is the genetic signature, the threshold level is suitably the sum of the expression levels of the control signature plus one standard deviation for the control signature.

A cancer cell with a TBRS+ signature that is also estrogen receptor negative is more likely to metastasize. Therefore, it would be useful to test a cancer cell that is TBRS+ for the presence of estrogen receptor.

Another embodiment of the invention is a subset of the 153 genes listed in Table 2 comprising 50 genes (Table 3), the expression levels of those 50 genes, when used in combination, have been found to allow correlation with the risk of lung metastasis in ER− cancer patients that are positive for LMS (Table 1), or that have tumors larger than 2 cm or that are positive for the wound signature or that have a basal subtype of ER− tumors.

Another embodiment of the invention is a subset of the 50 genes listed in Table 3 comprising 20 genes (Table 4), the expression levels of those 20 genes, when used in combination, have been found to allow correlation with the risk of lung metastasis in ER− cancer patients that are positive for LMS, or that have tumors larger than 2 cm or that are positive for the wound signature or that have a basal subtype of ER− tumors.

Another embodiment of the invention is a subset of at least 5 genes out of the 50 genes listed in Table 3 the expression levels of which, when used together, have been found to correlate with the risk of lung metastasis in ER− cancer patients that are positive for LMS, or that have tumors larger than 2 cm or that are positive for the wound signature or that have a basal subtype of ER− tumors. Many different gene sets of less than the 50 genes of Table 2 can be generated from the group of 50 genes in the TBRS-50, as many of these 50 genes individually show a p value of <0.05 in the correlation of their expression with TGFβ responsiveness. Tables 4 and 5 list different TBRS signatures with associated p values. It should be noted that certain genes are more commonly used in these signatures with high correlation levels. For example, BHLHB2, COL4A1 are used in five out of the six panels, CCDC93, JAG1, JUN, NR2F2, RAI2, RBMS1, ZFP36L1 are all used in four out of the six, and AGXT2L1, ALOX5AP, C6orf145, FAT4, FHL3, GADD45B, HMOX1, SERPINE1, SMTN, SMURF1, SPSB1, TNFRSF12A are used in three out of the six.

The expression levels of the genes used in the signatures of the present invention can be determined with commercially available test materials, as described below. However, it will be appreciated that the specific method of determination is not critical and that any method may be used. When a new method or platform for determining gene expression levels is used, a new standard curve for establishing TBRS positively and negativity of a sample has to be established using a large number of tumor samples, the number of tumor samples needed depending on the level of statistical confidence required.

Another embodiment of the present invention is a method to determine if a cancer patient would benefit from anti TGFβ therapy, the method comprising determining whether the cancer cells from that patient are TBRS positive or negative using any of the gene signatures of the present invention. A TBRS positive result makes the patient likely to benefit from anti TGFβ therapy.

The ER− tumors that are independently assessed to be LMS+ and TBRS+ are determined to have higher risk (50%) of lung metastasis. Therefore, it would be useful to test a TBRS+ cancer cell for LMS as well. In addition, it would be useful to test a TBRS+ cancer cell for LMS and estrogen receptor.

If a cancer is identified as one that has a high risk for metastasis to the lungs, management of this patient can take a more defined approach and involve:

(1) more aggressive treatment because the tumor has a higher risk of metastasis;

(2) more frequent follow-ups, with a focus on diagnostic imaging procedures in the lung

Since there appears to be a link between responsiveness to TGFβ and metastatic potential, the genes comprising the TGFβ response signature or their encoded proteins, are suitable targets for therapy. Therapy can be achieved by reducing the expression of an overexpressed gene, or by directly inhibiting the expressed protein. Targeting of one or more of these genes, for example using antisense or RNAi techniques could reduce lung metastasis activity.

Several studies have probed the link between TGFβ and lung metastasis. The study that is the basis of this invention sought to clarify the link between TGFβ responsiveness and metastatic potential. Using gene expression analysis, it was found that there is a definite connection between response to TGFβ and probability of metastasis to the lungs but not to other organs such as bone or liver. The basis for this connection seems to be TGFβ induction of angiopoietin-like 4 (ANGPTL4) in cancer cells that are about to enter the circulation which enhances their subsequent retention in the lungs

Moreover, the TBRS status of a tumor can be used to determine which cancer patient should be treated with therapies aimed at reducing or eliminating TGFβ pathway signaling. If a cancer patient is determined to be TBRS+ using any of the gene signatures of the present invention then that patient can be said to be eligible for, and have a high chance of benefiting from, such anti TGFβ therapy or therapies.

### Kit for Diagnosing Metastatic Potential for Cancer Cells

A kit may be used to determine metastatic potential. The kit contains reagents for determining expression levels of at least five markers selected from the group of genes typifying the TGFβ response in human epithelial cells. This kit may contain a gene chip with a set five or more markers selected from the group of genes typifying the TGFβ response in human epithelial cells. The gene chip could be an Affymatrix chip that detects the expression level of the desired markers.

The kit may also contain a plate with wells for determining the expression level of five or more markers. This plate would have separate wells with reagents for each marker.

### Development of a TGFβ Response Bioinformatics Classifier

In order to investigate the role of TGFβ in cancer progression, we set out to develop a bioinformatics classifier that would identify human tumors containing a high level of TGFβ activity. A gene expression signature typifying the TGFβ response in human epithelial cells was obtained from transcriptomic analysis of four human cell lines. These cell lines include HaCaT keratinocytes, HPL1 immortalized lung epithelial cells, MCF10A breast epithelial cells, and MDA-MB-231 breast carcinoma cells. The cells were treated with TGFβ1 for 3 h in order to capture direct TGFβ gene responses (Kang et al., 2003a). The resulting 153-gene TGFβ response signature (TBRS) (174 probe sets; Table 2) was used to generate a classifier by means of “meta-gene” analysis with the cell lines as references (Bild et al., 2006). The meta-gene analysis resulted in a continuous variable ranging from 0 to 1 that designates the relative level of TGFβ pathway activity in tissue samples. Using 0.5 as a threshold, most tumors could be unambiguously assigned to a TBRS− class or a TBRS+ class. When applied to metastatic lesions extracted from bones, lungs and other sites representing the natural metastatic spectrum of human breast cancer, the TBRS classifier identified TGFβ activity in a 38/67 of these samples (Table 6), which is in agreement with previous observations of activated Smad in a majority of human bone metastasis samples (Kang et al., 2005).

Development of a TGFβ Response Bioinformatics Classifier with Smaller Gene Sets

Among the 153 genes, 50 are univariately correlated with lung metastasis in ER− tumors. This is determined by the following procedures:
1) Find the express values of each of the 153 genes in each of the tumor microarray;
2) Find the lung metastasis incidences and the length of lung metastasis-free-survival for each corresponding patient (from whom the tumor was resected);
3) Fit this information in a Cox proportional hazards regression model (to examine whether there is significant correlation between the expression of these genes with lung metastasis incidences or length of lung metastasis-free survival);
4) The 50 individual genes that yielded significant correlations (p<0.05) were collected to form the TBRS50.

Combinations of any 5 or more genes from TBRS50 can also predict the risk of lung metastasis in ER− cancer patients. To try a particular combination (hereafter denoted as TBRS-x) from TBRS50 or from the original TBRS153, follow the procedures below:
1) Find the expression values of the genes in TBRS-x in each of the ER− tumors;
2) Hierarchically cluster the tumors based on the gene expression values of TBRS-x. We used R statistical software, “gplots” package and “heatmap.2” command, with the default parameter setting.
3) A cluster of tumors that overexpress all the upregulated genes in TBRS-x and underexpress all the downregulated genes in TBRS-x can be readily distinguished from the rest of tumors. And the tumors in this cluster are called TBRS+ and the rest called TBRS−;
   TBRS+ and TBRS− tumors were compared in terms of number of lung metastasis incidences and the length of lung metastasis-free survival in the corresponding ER− patients. A p value is calculated based on log-likelihood to denote the significance of the difference. We used R statistical software, “survival” package and “survdiff” command to implement this comparison.

By using this protocol any expert in the art can find gene signatures that can determine statistically significant TBRS that can be used in the present invention. As an example, but without being limited, Table 5 lists 5 different TBRS signatures (and its associated p values) that can be used in the present invention. Ideally one would choose a signature with the highest p value but in choosing a specific signature other considerations might be more important, for example a signature that uses few genes to be able to create simpler or cheaper signatures that are more easily incorporated into a commercial product.

Hierarchical clustering was performed on the MSK/EMC cohort with the indicated pathological and genomic markers including the TBRS, the lung metastasis signature (LMS), the wound response signature (Wound), the 70-gene prognosis signature (70-gene), size (Size>2 cm), the basal molecular subtype (Basal), and the ER status.

### TGFβ Activity in Primary Breast Tumors is Selectively Linked to Lung Metastasis

We applied the TBRS classifier to a series of primary breast carcinomas that were analyzed on the same microarray platform (Minn et al., 2007; Minn et al., 2005; Wang et al., 2005). This series includes 82 tumors collected at Memorial Sloan-Kettering Cancer Center (MSK cohort) and 286 tumors from the Erasmus Medical Center (EMC cohort). Both cohorts comprised a mix of breast cancer subtypes, with tumors in the MSK cohort being more locally advanced than those in the EMC cohort (Minn et al., 2007). Out of a combined total of 368 patients, 39 patients developed lung metastases and 83 developed bone metastasis after a median follow-up of 10 years, with some patients developing metastasis in both sites. TBRS+ tumors were similarly distributed between estrogen receptor-positive (ER+) and ER− tumors. Microarray analysis revealed that the TBRS+ tumors expressed significantly higher mRNA levels for TGFα1, TGFβ2, and the latent TGFβ activating factor, LTBP1. TBRS− tumors had lower mRNA levels for type II TGFβ receptor, Smad3 and Smad4. The expression level of other TGFβ pathway components was independent of TBRS status.

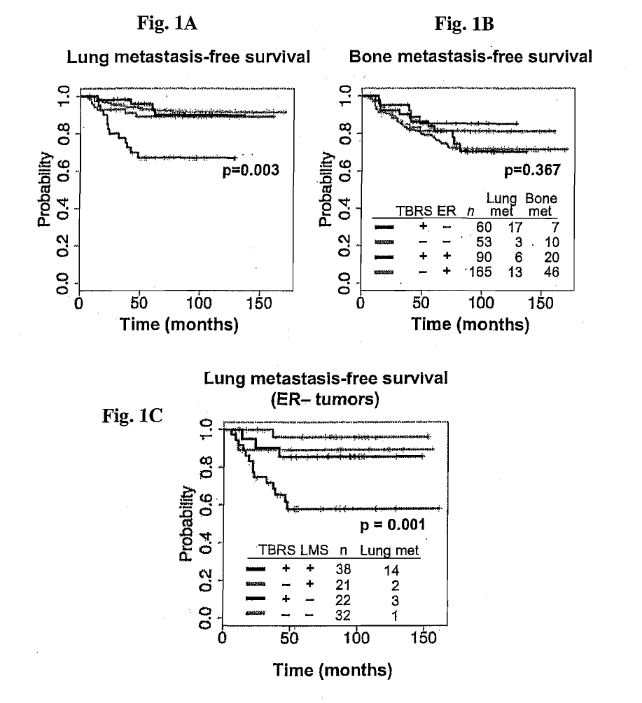

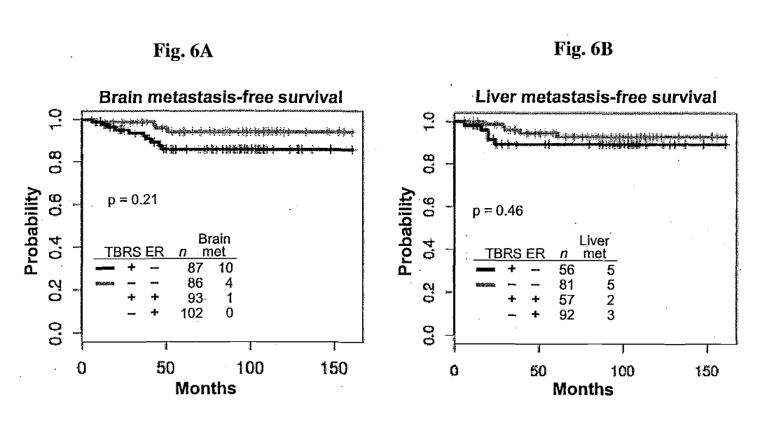

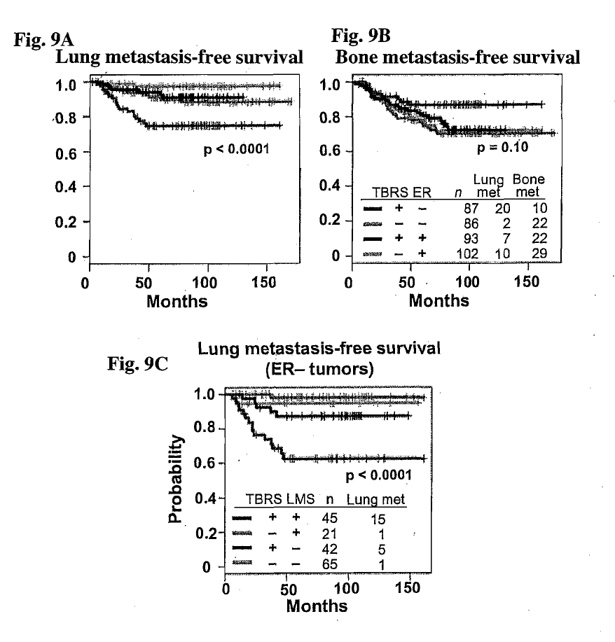

TBRS status in ER+ tumors did not correlate with distant metastasis. However, in ER− tumors there was a striking association between TBRS+ status and relapse to the lungs (FIG. 1A). This association was observed regardless of whether the tumor ER status was assigned using the clinical pathology reports, which are based on immunohistochemical analysis (FIG. 1A), or using a microarray probe level designation (FIG. 9A). No link was observed between TBRS status and bone metastasis (FIGS. 1A, 9A) or liver metastasis, and a brain metastasis association did not attain statistical significance (FIG. 6). In univariate as well as multivariate analyses, the expression level of TGFβ pathway components was much inferior to the TBRS at linking these tumors with metastasis outcome (Table 7). These results indicate that TGFβ activity in ER− breast tumors is selectively associated with lung metastasis.

### Cooperation Between TGFβ and the Lung Metastasis Signature

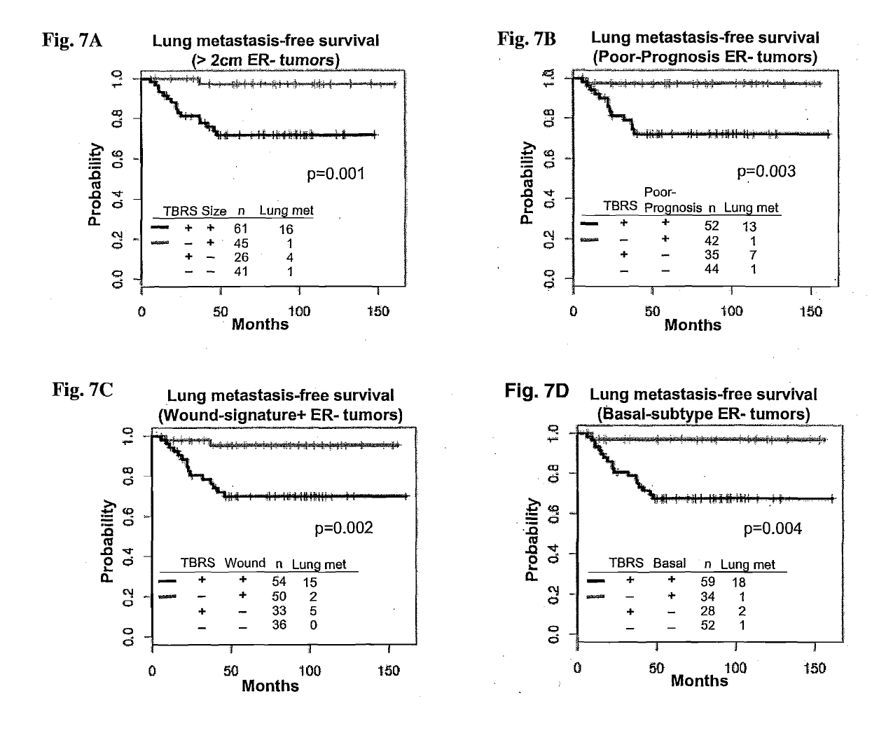

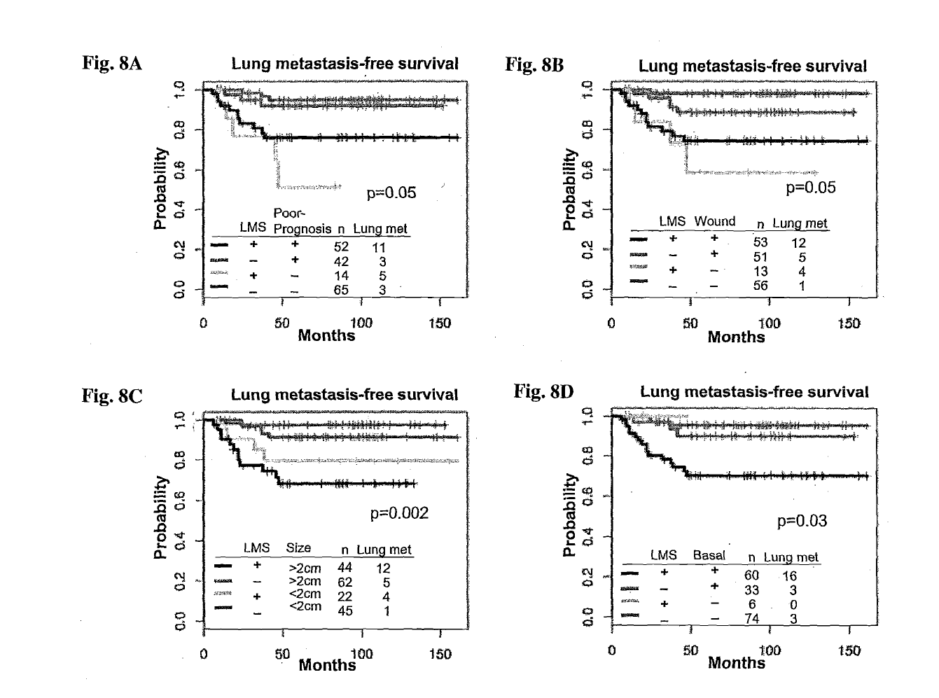

The association of TBRS with lung relapse prompted us to search for links between the TBRS and a previously described lung metastasis signature (LMS) (Minn et al., 2005). The LMS is a set of 17 genes whose expression in ER− tumors indicates a high risk of pulmonary relapse in patients (Minn et al., 2007). Several of these genes have been validated as mediators of lung metastasis (Gupta et al., 2007a; Gupta et al., 2007b; Gupta, 2007; Minn et al., 2005). The TBRS+ subset of ER− tumors partially overlapped the LMS+ subset. Remarkably, tumors that were positive for both the TBRS and LMS were associated with a high risk of pulmonary relapse, whereas single-positive tumors were not (FIG. 1B). Within poor-prognosis tumor subsets defined by other features, such as size>2 cm, basal subtype gene-expression signature (Sorlie et al., 2003), 70-gene poor prognosis signature (van de Vijver et al., 2002), or wound signature (Chang et al., 2005), TBRS status was associated with risk of lung metastasis in nearly every case. The TBRS performed independently of these other prognostic features (FIG. 7), as did the LMS (FIG. 8 (Minn et al., 2007).

### TGFβ Signaling in Mammary Tumors Enhances Lung Metastatic Dissemination

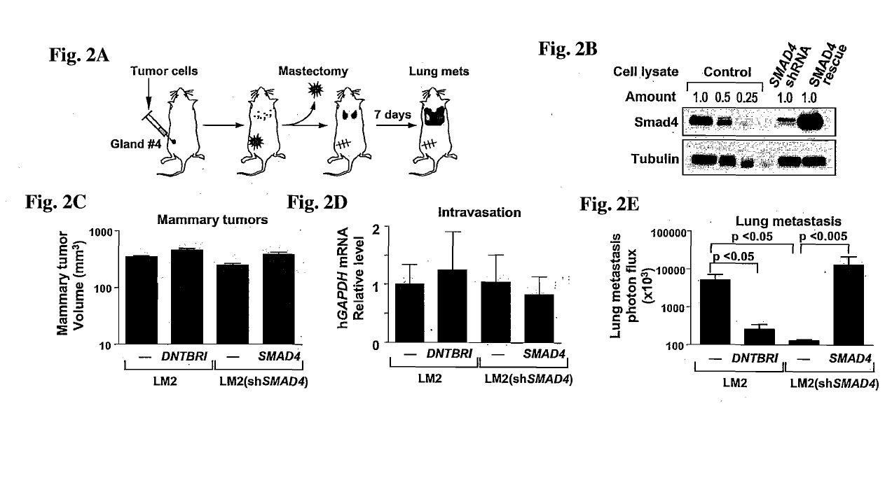

To functionally test whether TGFβ signaling in primary tumors contributes to lung metastases, we used a xenograft model of ER− breast cancer metastasis (Minn et al., 2005). The MDA-MB-231 cell line was established from the pleural fluid of a patient with ER− metastatic breast cancer (Cailleau et al., 1978). MDA-MB-231 cells have a functional Smad pathway and evade TGFβ growth inhibitory responses though alterations downstream of Smads (Gomis et al., 2006). The lung metastatic subpopulation LM2-4175 (henceforth LM2) was isolated by in vivo selection of MDA-MB-231 cells (Minn et al., 2005). We perturbed the TGFβ pathway in LM2 cells by overexpressing a kinase-defective, dominant-negative mutant form of the TGFβ type I receptor (Weis-Garcia and Massagué, 1996), or by reducing the expression of Smad4, which is an essential partner of Smad2/3 in the formation of transcriptional complexes (Massaguéet al., 2005). Using a validated SMAD4 short-hairpin RNA (shRNA) (Kang et al., 2005) we reduced Smad4 levels by 80-90% in LM2 cells (FIG. 2B). As a control, we generated SMAD4 rescue cells by expressing a shRNA-resistant SMAD4 cDNA in SMAD4 knockdown cells (FIG. 2B).

Neither the dominant negative TGFβ receptor nor the Smad4 knockdown decreased mammary tumor growth as determined by tumor volume measurements, or the extent of tumor cell passage into the circulation, as determined by qRT-PCR analysis of human GAPDH mRNA in blood cellular fractions (FIGS. 2C, 2D). Tumors inoculated into the mammary glands of immunocompromised mice and allowed to grow to 300 mm³, were surgically removed and the emergence of distant metastases after the mastectomy was determined (FIG. 2A). Inactivation of TGFβsignaling markedly inhibited the lung metastatic activity of the tumors as determined by quantitative luciferase bio-luminescence imaging (FIG. 2E) (Ponomarev et al., 2004) and histological examination. These results suggest that the canonical TGFβ pathway enhances mammary tumor dissemination to the lungs.

TGFβ Primes Tumor Cells to Seed of Lung Metastases

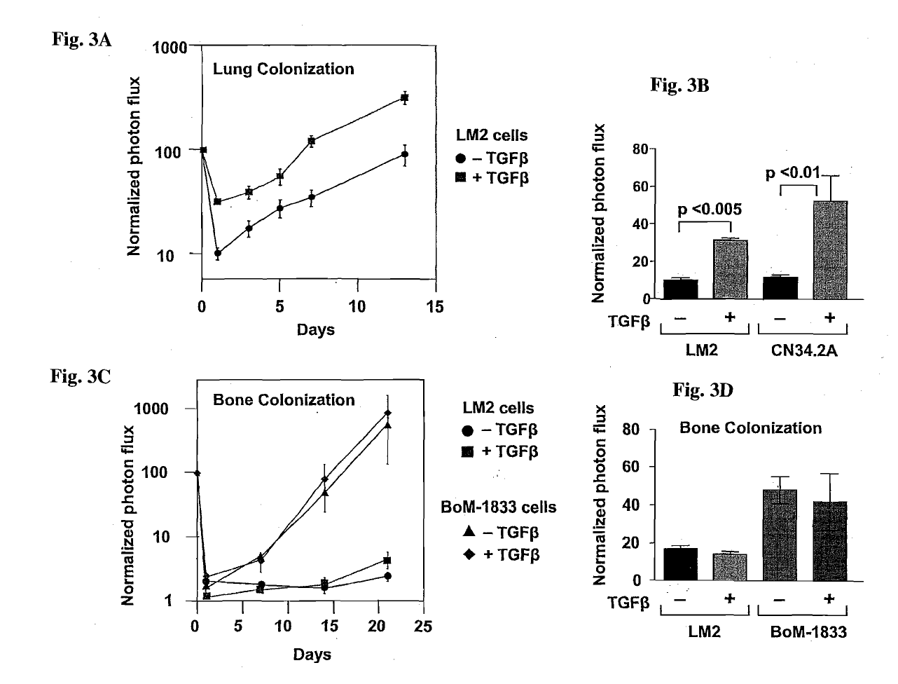

We wondered whether TGFβ within the tumor microenvironment could endow tumor cells with the ability to seed the lungs as these cells enter the circulation. To test this possibility, we mimicked the exposure of tumor cells to TGFβ by incubating LM2 cells with TGFβ for 6 h prior to inoculation of these cells into the tail vein of mice. Interestingly, this pre-treatment with TGFβ significantly increased the lung colonizing activity of LM2 cells, as determined by a higher retention of these cells in the lungs 24 h after inoculation (FIG. 3A). In this time frame LM2 cells extravasate into the lung parenchyma (Gupta et al., 2007a). A similar effect was observed when we carried out this experiment with malignant cells (CN34.2A) obtained from the pleural fluid of a breast cancer patient treated at MSKCC. The pre-treatment with TGFβ increased the lung seeding activity of LM2 and CN34.2A cells three- and five-fold, respectively (FIG. 3B). The initial advantage provided by a transient exposure to TGFβ was sustained but not expanded during the ensuing outgrowth of metastatic colonies (FIG. 3A).

To investigate the selectivity of this lung metastasis-priming effect, we tested the effect of TGFβ pre-incubation on the establishment of bone metastases. LM2 cells have limited bone metastatic activity in addition to their high lung metastatic activity (Minn et al., 2005). The pre-treatment of LM2 cells with TGFβ prior to their inoculation into the arterial circulation did not increase the ability of these cells to form bone metastases (FIG. 3C). We also tested the effect of TGFβ on the metastatic activity of an MDA-MB-231 sub-population (BoM-1833) that is highly metastatic to bone (Kang et al., 2003b) and responsive to TGFβ (Kang et al., 2005). Pre-incubation of BoM-1833 cells with TGFβ did not increase their ability to form bone metastases (FIG. 3C), and had no discernible effect on the early seeding of the bones (FIG. 3D). Thus, TGFβ stimulation primes tumor cells for an early step in lung metastasis but not bone metastasis, which is concordant with the selective association of TBRS+ status in primary tumors with risk of lung metastasis in clinical cohorts (refer to FIG. 1A).

### Assessment of TBRS Status

The assessment of TBRS status can be carried out in one of two ways. The first method involves the performance of a “meta-gene” analysis based on the TBRS50 gene set and using the cell lines as references (Bild et al., 2006). For each tumor, a number between 0 and 1 is derived, indicating the likelihood that the TGFβ signaling is active in that tumor. The tumor being tested is also assigned a score based on the gene expression of TBRS50 and thus TBRS status is determined. The second method involves clustering tumors with known TBRS50 expression levels based on these levels and identifying where a tumor from a patient with unknown TBRS status fits into this cluster map.

### Protocol for Assessing TBRS Status in a Patient with a Tumor Using Affymetrix Chips

### Example 1

### Protocol I, Metagene Analysis

The tumor sample is profiled with Affymetrix U133Plus2 or U133a chips, using 5 ug RNA and the standard protocol recommended by the manufacturer. The data are pre-processed using RMA algorithm (available in affy package). The median of expression values of all genes are set to 0. The expression values of TBRS50 are found. The cell line data are merged with the patient data to get a matrix of 13×50 (designated as tbrs hereafter), where there are 13 columns (12 cell lines plus one patient) and 50 rows (gene expression values of the TBRS50 genes). Principle component analysis (PCA) is performed on the matrix (using command prcomp(t(tbrs)), default setting). The first principle component of the 12 cell lines derived from above analysis is used to train a Bayesian probit model using the package MCMCpack, command MCMCprobit, with parameters: thin=100, burnin=100000, mcmc=100000, seed=6032005. The first principle component of the patient (derived from step 8) is fit to the probit model in the above step, using the script in appendix.in R. A score between 0 and 1 will be generated. A score above 0.5 can be considered as positive for the TBRS.

### Protocol II, Unsupervised Clustering:

The tumor sample is profiled with Affymetrix U133Plus2 or U133a chips, using 5 ug RNA and the standard protocol recommended by the manufacturer. The data are pre-processed using RMA algorithm (available in affy package). The median of expression values of all genes are set to 0. The expression values of TBRS50 are found. The MSK and Erasmus datasets are obtained from the GEO database (accession numbers are GSE2603 and GSE2034, respectively). The two datasets are combined. The tumor data are normalized the same way as above. The subset patients with negative ER status are located (annotations available with the datasets at the GEO website). The TBRS50 in ER− patients are obtained. The datasets from the 368 tumors are combined with the data from the patient with the unknown TBRS status to get a matrix of N+1 columns and 50 rows (N=number of ER− patients). The matrix is clustered using command heatmap.2 in package gplots. For better visualization, the following parameters can be used: scale=“row”, col=greenred(100), trace=“none”. Two major clusters will be revealed. One will display significantly higher expression of the vast majority of the TBRS and will be designated as TBRS+. The other cluster will be denoted as “TBRS-”. The patient with unknown TBRS status will be located on this map and it will be possible to determine the cluster at which it is located. The TBRS status of this patient will be determined according to the cluster it resides.

Note: All algorithms were implemented in R statistical software.

### Script for Fitting the MCMCprobit Model:

```
predict.mean <- function(A, B)
{
    A <- as.matrix(A)
    I <- dim(A)[1]
    y <- rep(NA, I)
    for(i in 1:I)
    {
        x <- c(1, A[i,])
        eta <- as.vector(x)%*%t(as.matrix(B))
        p <- pnorm(eta)
        y[i] <- mean(p)
    }
    y
}
A: principal components of the patient;
B: the MCMCprobit model.
```

Protocol for Assessing TBRS Status in a Patient with a Tumor Using Hybridization Technique Other than Affymetrix Chips

Each time a new method of determining the RNA levels for TBRS50 is used, a new standard curve has to be set up first to estimate the distribution of each gene among patients. A gene could be easily detected using hybridization based technology or with polymerase chain reaction technology, but the absolute intensity of signal in a single patient can be influenced by many factors, including the efficiency of hybridization, the amount and quality of total RNA loaded, etc. All these need to be normalized using a statistically significant number of patients, preferentially with known clinical outcomes (just like points on a standard curve). Then additional patients could be interrogated and compared with the standard curve. Then the prognosis can be made.

### Combining TBRS and LMS Signatures for Better Prediction of Lung Metastasis Prognosis

When the same set of tumors was tested for TBRS positively and LMS positively, there was a subset of ER− tumors that tested positive for both. Remarkably, tumors that were positive for both the TBRS and LMS were associated with a high risk of pulmonary relapse, whereas single-positive tumors were not. So, for a more refined approach towards predicting lung metastasis potential of cancers, the patient sample can be tested for TBRS as well as LMS status. The tumors that test positive for both signatures can be treated with a view to minimizing future lung metastasis.

### Example 1

### TGFβ Response Gene-Expression Signature and TBRS Classifier

Cell lines with and without TGFβ1 treatment (3 h, 100 μM) were subject to expression profiling using Affymetrix U133A or U133 plus2 microchips. Microarray results were pre-processed using RMA algorithm (carried with affy package of R statistical program). The first comparison was conducted between all TGFβtreated samples versus all untreated samples. Three hundred and fifty genes that yielded a p value of 0.05 or less (after Benjamini and Hochberg correction for multiple tests) were kept. Among these genes, we chose to focus on the genes that are significantly changed in at least two different cell lines when the cell lines are considered separately. This step resulted in 174 probe sets corresponding to 153 distinct human genes, which were collectively designated as the TGFβ gene response signatures.

To generate a TBRS classifier, we carried out a “meta-gene” analysis based on this gene set and using the cell lines as references (Bild et al., 2006) and references therein. In short, expression values of the 153 TGFβ responsive genes in cell lines were linearly transformed and encapsulated into one or two “Meta genes”. A Bayesian Probit model was then trained based the cell line data and applied to the Meta genes of the tumor samples. For each tumor, a number between 0 and 1 was derived, indicating the likelihood that the TGFβ signaling is active in that tumor.

### Example 2

### Cell Culture and Reagents

MDA-MB-231 and its metastatic derivatives LM2-4175 and BoM-1833 have been described previously (Kang et al., 2003b; Minn et al., 2005). Breast carcinoma cells were isolated from the pleural effusion of patients with metastatic breast cancer treated at our institution upon written consent obtained following IRB regulations as previously described (Gomis et al., 2006). BCN samples were obtained and treated as per Hospital clinic de Barcelona guidelines (CEIC-approved).

TGFβ and TGFβ-receptor inhibition used 100 pM TGFβ1 (R&D Systems) for 3 or 6 h as indicated and 10 μM SB431542 (Tocris) with 24 h pretreatment. Epithelial cell lines were treated for 3 h with BMP2 (25 ng/mL, R&D), Wnt3a (50 ng/mL, R&D), FGF (5 ng/mL, Sigma), EGF (100 ng/mL, Invitrogen), IL6 (20 ng/mL, R&D), VEGF-165 (100 ng/mL, R&D), and IL1β (100 ng/mL, R&D). Conditioned media experiments were performed by growing cells in serum-deprived media for 48 hours. Recombinant human Angpt14 (Biovendor) was used at 2.5 μg/mL for 24 h.

### Example 3

### RNA Isolation, Labeling, and Microarray Hybridization

Methods for RNA extraction, labeling and hybridization for DNA microarray analysis of the cell lines have been described previously (Kang et al., 2003b; Minn et al., 2005). The EMC and MSK tumor cohorts and their gene expression data have been previously described (Minn et al., 2007; Minn et al., 2005; Wang et al., 2005). Bone or lung recurrence at any time is indicated.

### Example 4

### Generation of Retrovirus and Knockdown Cells

Knockdown of SMAD4 and ANGPTL4 was achieved using pRetroSuper technology (Brummelkamp et al., 2002) targeting the following 19-nucleotide sequences: 5′-GGTGTGCAGTTGGAATGTA-3′ (SEQ. ID No. 1) (SMAD4) and 5′-GAGGCAGAGTGGACTATTT-3′ (SEQ. ID No. 2) (ANGPTL4). To produce retrovirus for knockdown, the hairpin vector was transfected into the GPG29 amphotropic packaging cell line (Ory et al., 1996).

### Example 5

### Immunofluorescence

HUVECs were grown to confluence on fibronectin coated chamber slides (BD Biosciences). The cells were fixed for 10 min in 4% paraformaldehyde in PBS, and incubated for 5 min on ice in 0.5% Triton X-100 in PBS. After blocking with 2% BSA, the monolayers were processed for staining with anti-ZO1 (Zymed), anti-beta-catenin (Santa Cruz), rhodamine phalloidin (Molecular Probes) for F-actin staining and DAPI (Vector Labs) for nuclear staining. Fluorescence images were obtained using an Axioplan2 microscopy system (Zeiss).

### Example 6

### Animal Studies

All animal work was done in accordance with a protocol approved by the MSKCC Institutional Animal Care and Use Committee. NOD/SCID female mice (NCI) age-matched between 5-7 weeks were used for xenografting studies. For experimental metastasis assays from orthotopic inoculations, the tumors were extracted from both mammary glands when they each reached 300 mm³, approximately 30 days. Seven days after mastectomies, lung metastases were monitored and quantified using non-invasive bioluminescence as previously described (Minn et al., 2005).

### Example 7

### In Vivo Lung Permeability Assays

To observe in vivo permeability of lung blood vessels, tumor cells were labeled by incubating with 5 μM cell tracker green (Invitrogen) for 30 min and inoculated into the lateral tail vein. One day post inoculation, mice were injected intravenously with rhodamine-conjugated dextran (70 kDa, Invitrogen) at 2 mg per 20 g body weight. After 3 h, mice were sacrificed; lungs were extracted and fixed by intra-tracheal injection of 5 mL of 4% PFA. Lungs were fixed-frozen and 10 μm sections were taken to be examined by fluorescence microscopy for vascular leakage. Images were acquired on an Axioplan2 microscopy system (Zeiss). To analyze, a uniform ROI of approximately 3 nuclei in diameter was drawn around the tumor cells and applied to each image. A second larger ROI was also applied with similar results. Signal from the ROI was quantified using Volocity (Improvision).

### Example 8

### Statistical Analysis

Results are reported as mean±standard error of the mean unless otherwise noted. Comparisons between continuous variables were performed using an unpaired one-sided t-test. Statistics for the orthotopic lung metastasis assays were performed using log-transformation of raw photon flux.

### Example 9

### Cell Culture and Reagents

HaCaT were maintained in DMEM medium supplemented with 10% fetal bovine serum (FBS), penicillin, streptomycin, and fungizone. MCF-10A cells were maintained in a 1:1 mixture of DMEM and Ham's F12 supplemented with 5% horse serum, 10 μg/ml insulin (Sigma), 0.5 μg/ml hydrocortisone (Sigma), 0.02 μg/ml epidermal growth factor (Sigma), and antibiotics. HPL1 cells were maintained in Ham's F12 supplemented with 1% FBS, 5 μg/ml insulin, 0.5 μg/ml hydrocortisone, 5 μg/ml transferrin (Sigma), 2×10¹⁰M triiode thyronine, and antibiotics. All tumor cell lines were cultured in DMEM supplemented with 10% FBS, glutamine, penicillin, streptomycin and fungizone. The pleural effusion samples were centrifuged at 1,000 r.p.m. for 10 min, cell pellets were re-suspended in PBS and treated with ACK lysis buffer to lyse blood cells. A fraction of these cells underwent negative selection to remove leukocytes (CD45⁺ and CD15⁺ cells), and EpCam-positive cells were sorted from the population upon recovery in tissue culture for 24 h. Human vascular endothelial cells (HUVECs) (ScienCell) were cultured in complete ECM media (ScienCell) and used between passages 3-6. The retroviral packaging cell line GPG29 was maintained in DMEM containing 10% FBS supplemented with puromycin, G418, doxycycline, penicillin, streptomycin and fungizone. Transfections were done using standard protocols with Lipofectamine 2000 (Invitrogen). After transfection, GPG29 cells were cultured in DMEM containing 10% FBS.

### Example 10

### RNA Isolation, Labeling, and Microarray Hybridization

The tissues for microarray analysis were obtained from therapeutic procedures performed as part of routine clinical management. Samples were snap-frozen in liquid nitrogen and stored at −80 C. Each sample was examined histologically in cryostat sections stained with hematoxylin and eosin. Regions were dissected manually from the frozen block to provide a consistent tumor cell content of greater than 70% in tissues used for analysis. RNA was extracted from frozen tissues by homogenization in TRIzol reagent (Gibco/BRL) and evaluated for integrity. Complementary DNA was synthesized from total RNA by using a dT primer tagged with a T7 promoter. The RNA target was synthesized by transcription in vitro and labeled with biotinylated nucleotides (Enzo Biochem). The labeled target was assessed by hybridization to Test3 arrays (Affymetrix). All gene expression analysis was performed with an HG-U133A GeneChip (Affymetrix). Gene expression was quantified with MAS 5.0 or GCOS (Affymetrix). All studies involving patient materials or data were conducted under protocols approved by the Institutional Review Board of Memorial Sloan-Kettering Cancer Center, and that of the Hospital Clinic de Barcelona.

For the cell culture experiments, total RNA was prepared from 5×10⁶ cultured cells that were untreated or treated with TGFβ. Twenty-five micrograms of total RNA was used to prepare cRNA probe using a Custom Superscript Kit (Invitrogen) and the BioArray HighYield RNA Transcript Labeling Kit (Enzo). Each sample was hybridized with an Affymetrix Human Genome U133A microarray for 16 hr at 45° C.

### Example 11

### Generation of Retrovirus and Knockdown Cells

Viruses were collected 48 and 72 h after transfection, filtered, and concentrated by ultracentrifugation. Concentrated retrovirus was used to infect cells in the presence of 8 μg ml⁻¹ polybrene, typically resulting in a transduction rate of over 80%. Infected cells were selected with puromycin or hygromycin. To generate knockdown-rescue cell lines, we used a similar method to produce virus encoding complementary DNAs for overexpression of the RNAi-targeted genes, along with a hygromycin or puromycin selectable marker. The overexpressing retrovirus vector, pBabe, was used to super-infect previously generated knockdown cells that were subsequently selected with either hygromycin or puromycin.

### Example 12

### Analysis of mRNA and Protein Expression

Total RNA from subconfluent MDA-MB-231 cells was collected and purified using the RNeasy kit (Qiagen). Four-hundred nanograms of total purified RNA was subjected to a reverse transcriptase reaction according to the Hi-Capacity Archive kit (Applied Biosystems). cDNA corresponding to approximately 4 ng of starting RNA was used in three replicates for quantitative PCR. Indicated Taqman gene expression assays (Applied Biosystems) and the Taqman universal PCR master mix (Applied Biosystems) were used to quantify expression. Quantitative expression data were acquired and analyzed using an ABI Prism 7900HT Sequence Detection System (Applied Biosystems). For immunoblotting, we used previously described methods (Calonge and Massagué, 1999). Briefly, proteins were separated by SDS-PAGE and transferred to nitrocellulose membranes (Bio-Rad) that were immunoblotted with mouse monoclonal antibodies that recognize Smad4 (Cell Signaling) and α-tubulin (Sigma). For analysis of secreted protein expression, cells were plated in triplicate at 90% confluency in 12-well plates, incubated in DMEM 0.2% FBS, and conditioned media was collected 72 h later. Media was cleared of cells by centrifuging at 2,000 r.p.m. for 5 min. Angpt14 concentrations were analyzed in conditioned media using an ELISA kits (BioVendor).

### Example 13

### Immunostaining

For visualizing lung metastases, mice were killed and perfused with PBS and 4% paraformaldehyde through the left ventricle. Lungs were fixed and paraffin-embedded. Immunohistochemical staining for vimentin (Novocastra) was performed on paraffin-embedded lung sections by the MSKCC Molecular Cytology Core Facility. Brightfield microscopic images were collected using an Axioplan2 microscopy system (Zeiss).

### Example 14

### Animal Studies

For primary tumor analysis, 5×10⁵ viable single cells were re-suspended in a 1:1 mixture of PBS and growth-factor-reduced Matrigel (BD Biosciences) and injected orthotopically into both mammary gland number four in a total volume of 100 μL as previously described (Minn et al., 2005). Primary tumor growth rates were analyzed by measuring tumour length (L) and width (W), and calculating tumor volume based on the formula IILW²/6. For lung colonization assays, 2×10⁵ cells were re-suspended in 0.1 ml PBS and injected into the lateral tail vein. Lung metastatic progression was again monitored and quantified using bioluminescence. For bone metastasis, 30,000 cells in PBS were injected into the left ventricle of anaesthetized mice (100 mg kg⁻¹ ketamine, 10 mg kg⁻¹ xylazine). For priming assays, cells were switched to low media (0.2% FBS) for 12 hours and then treated with 100 μM of TGFβ for 6 hours. Mice were inoculated with 200,000 and 30,000 cells for lung and bone assays, respectively. Mice were imaged for luciferase activity immediately after injection to exclude any that were not successfully xenografted. Lymph node analysis was performed by ex vivo bioluminescent imaging of the peri-aortic lymph nodes.

### TBRS in Metastatic Melanomas

Tissue samples were taken from patients with metastatic melanomas. The tissues were taken from skin or lymph node metastases of melanomas. The data analyzed are publicly available at Gene Expression Omnibus (GEO) database, accession number is GSE8401. The clinical annotations of these patients were published in Xu et al., 2008, Mol. Cancer Res., 6(5): 760-769.

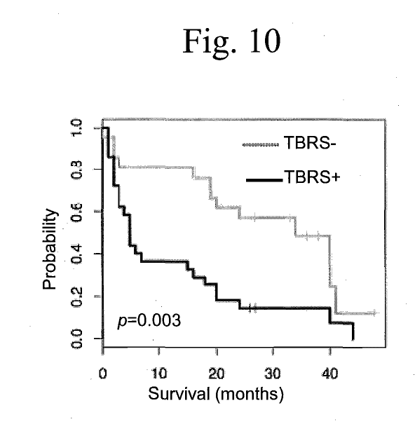

FIG. 10 shows Kaplan-Meier curves obtained using the 153 gene signature. TBRS was applied to 52 metastatic malignant melanomas using the same approach described in Padua et al., 2008 and the previous section of this application. Patients were classified into two groups. The group with a positive pattern of TBRS expression (TBRS+) exhibits worse overall survival compared to TBRS− group, as shown by the Kaplan-Meier curves (median survival 5 months vs. 40 months).

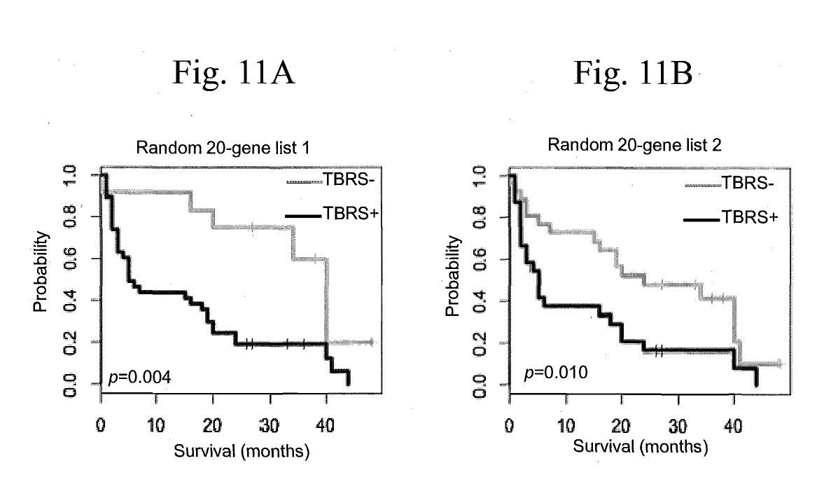

FIGS. 11A and 11B show Kaplan-Meier curves obtained using 20 gene signatures. FIG. 11A used the genes listed in Run 1 of Table 5 and FIG. 11B used the genes listed in Run 2 of Table 5. Again, the group with a positive pattern of TBRS expression (TBRS+) exhibits worse overall survival compared to TBRS− group.

### TBRS in Colon Cancers

TBRS was applied to patients in accession number GSE5206. 35 patients are classified as TBRS+, among which 11 developed recurrences (31.4%). 65 patients are classified as TBRS−, among which only 1 patient developed recurrence (1.54%). The difference is highly significant (p=2.7e-5, Fisher's Exact Test). The description of GSE5206 can be found together with the microarray data at GEO: http://www.ncbi.nlm.nih.gov/geo/query/acc.cgi?acc=GSE5206. This shows that the TBRS+ signature is implicated in recurrence on colon cancer.

TABLE 1
17-gene classifier (LM17) to predict the expression of the lung metastasis
signature.
Shown are the genes used in class prediction. In leave-one-out cross
validation, support vector machine yielded a 97% correct classification rate, with a
permutation p-value of less than 0.00005. Shown for each gene is the p-value to test the
null hypothesis that the gene is differentially expressed between the classes used during
training.

| | Parametric | | | Gene |
|---|---|---|---|---|
| | p-value | Description | GB acc | symbol |
| 1 | <0.000001 | singed (Drosophila)-like (sea urchin fascin | NM_003088 | FSCN1 |
| | | homolog like) | | |
| 2 | <0.000001 | matrix metalloproteinase 1 (interstitial collagenase) | NM_002421 | MMP1 |
| 3 | <0.000001 | PPAR (gamma) angiopoietin related protein | NM_016109 | ANGPTL4 |
| 4 | 0.000006 | adipose specific 2 | NM_006829 | APM2 |
| 5 | 0.00002 | GRO1 oncogene (melanoma growth stimulating | NM_001511 | CXCL1 |
| | | activity, alpha) | | |
| 6 | 0.000355 | prostaglandin-endoperoxide synthase 2 | NM_000963 | PTGS2 |
| | | (prostaglandin G/H synthase and cyclooxygenase) | | |
| 7 | 0.000444 | keratin, hair, basic, 1 | NM_002281 | KRTHB1 |
| 8 | 0.000506 | vascular cell adhesion molecule 1 | NM_001078 | VCAM1 |
| 9 | 0.000627 | retinoic acid receptor responder (tazarotene | NM_004585 | RARRES3 |
| | | induced) 3 | | |
| 10 | 0.001263 | latent transforming growth factor beta binding | NM_000627 | LTBP1 |
| | | protein 1 | | |
| 11 | 0.004365 | kynureninase (L-kynurenine hydrolase) | NM_003937 | KYNU |
| 12 | 0.005179 | chemokine (C—X—C motif), receptor 4 (fusin) | NM_003467 | CXCR4 |
| 13 | 0.006426 | lymphocyte antigen 6 complex, locus E | NM_002346 | LY6E |
| 14 | 0.007153 | inhibitor of DNA binding 1, dominant negative | NM_002165 | ID1 |
| | | helix-loop-helix protein | | |
| 15 | 0.010871 | mannosidase, alpha, class 1A, member 1 | NM_005907 | MAN1A1 |
| 16 | 0.032361 | enhancer of filamentation 1 (cas-like docking; Crk- | NM_006403 | NEDD9 |
| | | associated substrate related) | | |
| 17 | 0.03713 | epiregulin | NM_001432 | EREG |

TABLE 2
The 153 genes in the TBRS. Gene symbols, gene description, and TGFβ
induction levels are displayed.

| | MDA-MB- | | | | |
|---|---|---|---|---|---|
| Probes | 231 | HaCaT | HPL1 | MCF10A | Symbol |
| 213497_at | 1.88 | 2.14 | 2.24 | 1.53 | ABTB2 |
| 206466_at | 1.09 | 2.90 | 17.93 | 1.18 | ACSBG1 |
| 222090_at | 0.97 | 0.55 | 0.44 | 0.46 | ADCK2 |
| 206170_at | 0.37 | 0.40 | 0.73 | 0.52 | ADRB2 |
| 207294_at | 0.98 | 0.69 | 0.35 | 0.06 | AGTR2 |
| 221008_s_at | 1.01 | 0.76 | 0.15 | 0.34 | AGXT2L1 |
| 220842_at | 1.01 | 1.03 | 0.13 | 0.04 | AHI1 |
| 204174_at | 1.28 | 1.36 | 2.19 | 2.02 | ALOX5AP |
| 222108_at | 4.26 | 1.81 | 5.54 | 1.51 | AMIGO2 |
| 217630_at | 1.02 | 0.53 | 0.16 | 0.47 | ANGEL2 |
| 221009_s_at | 7.58 | 2.18 | 40.35 | 6.26 | ANGPTL4 |
| 207087_x_at | 1.02 | 1.23 | 7.13 | 4.36 | ANK1 |
| 217888_s_at | 2.23 | 1.02 | 1.45 | 2.01 | ARFGAP1 |
| 207606_s_at | 0.40 | 1.10 | 0.50 | 0.54 | ARHGAP12 |
| 212614_at | 0.40 | 0.41 | 0.31 | 0.42 | ARID5B |
| 217579_x_at | 1.04 | 1.69 | 9.19 | 2.77 | ARL6IP2 |
| 216197_at | 1.07 | 0.64 | 0.40 | 0.05 | ATF7IP |
| 218631_at | 0.49 | 0.76 | 0.42 | 0.37 | AVPI1 |
| 220470_at | 1.13 | 1.05 | 5.04 | 4.41 | BET1L |
| 201169_s_at | 3.67 | 2.21 | 9.39 | 2.27 | BHLHB2 |
| 201170_s_at | 2.99 | 3.37 | 7.51 | 2.45 | BHLHB2 |
| 206119_at | 1.15 | 1.66 | 2.13 | 4.44 | BHMT |
| 209920_at | 1.72 | 2.35 | 2.18 | 1.29 | BMPR2 |
| 210214_s_at | 2.22 | 1.45 | 2.14 | 2.61 | BMPR2 |
| 206787_at | 0.98 | 0.72 | 0.12 | 0.06 | BRDT |
| 218723_s_at | 0.50 | 0.56 | 0.05 | 0.24 | C13orf15 |
| 217508_s_at | 1.87 | 1.73 | 2.62 | 1.47 | C18orf25 |
| 221533_at | 1.18 | 1.18 | 2.04 | 3.22 | C3orf28 |
| 219474_at | 2.00 | 1.80 | 2.46 | 2.14 | C3orf52 |
| 212923_s_at | 2.12 | 2.21 | 1.61 | 1.52 | C6orf145 |
| 220513_at | 1.09 | 1.95 | 7.90 | 2.09 | C6orf148 |
| 209688_s_at | 1.63 | 1.36 | 2.07 | 2.30 | CCDC93 |
| 219774_at | 2.14 | 1.26 | 1.04 | 2.52 | CCDC93 |
| 203645_s_at | 0.96 | 0.76 | 0.36 | 0.15 | CD163 |
| 208592_s_at | 1.23 | 1.37 | 4.40 | 4.29 | CD1E |
| 211861_x_at | 1.09 | 0.68 | 0.48 | 0.08 | CD28 |
| 202284_s_at | 3.18 | 2.70 | 2.44 | 1.11 | CDKN1A |
| 218929_at | 0.59 | 0.96 | 0.37 | 0.47 | CDKN2AIP |
| 203973_s_at | 0.21 | 0.57 | 0.44 | 0.53 | CEBPD |
| 207331_at | 1.08 | 2.05 | 10.46 | 1.02 | CENPF |
| 207980_s_at | 0.12 | 0.34 | 0.17 | 0.23 | CITED2 |
| 209357_at | 0.13 | 0.19 | 0.22 | 0.19 | CITED2 |
| 205295_at | 1.11 | 0.65 | 0.47 | 0.23 | CKMT2 |
| 202310_s_at | 4.75 | 1.33 | 1.90 | 5.05 | COL1A1 |
| 211980_at | 2.07 | 1.47 | 2.64 | 4.02 | COL4A1 |
| 211981_at | 1.84 | 1.40 | 2.99 | 2.27 | COL4A1 |
| 211964_at | 1.27 | 1.19 | 2.08 | 2.08 | COL4A2 |
| 221900_at | 1.21 | 1.10 | 2.01 | 3.43 | COL8A2 |
| 209101_at | 4.01 | 5.88 | 2.12 | 5.20 | CTGF |
| 206775_at | 1.22 | 1.07 | 3.13 | 2.26 | CUBN |
| 203923_s_at | 1.13 | 0.76 | 0.07 | 0.19 | CYBB |
| 202887_s_at | 0.50 | 0.93 | 0.79 | 0.47 | DDIT4 |
| 215252_at | 1.12 | 1.56 | 2.08 | 3.67 | DNAJC7 |
| 40612_at | 0.94 | 0.55 | 0.15 | 0.30 | DOPEY1 |
| 218995_s_at | 3.76 | 2.13 | 1.50 | 1.03 | EDN1 |
| 206127_at | 2.01 | 1.56 | 2.11 | 1.35 | ELK3 |
| 221773_at | 2.09 | 1.90 | 3.03 | 1.62 | ELK3 |
| 201328_at | 1.72 | 3.35 | 2.86 | 6.83 | ETS2 |
| 201329_s_at | 1.75 | 2.65 | 1.72 | 3.95 | ETS2 |
| 219427_at | 0.61 | 0.70 | 0.50 | 0.39 | FAT4 |
| 204988_at | 1.01 | 0.55 | 0.18 | 0.36 | FGB |
| 218818_at | 2.21 | 1.19 | 1.60 | 2.51 | FHL3 |
| 204135_at | 2.28 | 1.38 | 1.23 | 2.44 | FILIP1L |
| 220326_s_at | 1.30 | 1.58 | 2.06 | 2.10 | FLJ10357 |
| 58780_s_at | 1.50 | 1.81 | 2.22 | 2.19 | FLJ10357 |
| 210316_at | 0.96 | 0.48 | 1.15 | 0.14 | FLT4 |
| 219316_s_at | 0.85 | 0.48 | 0.87 | 0.17 | FLVCR2 |
| 218618_s_at | 2.01 | 1.25 | 1.26 | 2.09 | FNDC3B |
| 203592_s_at | 1.90 | 2.20 | 3.24 | 1.71 | FSTL3 |
| 209414_at | 1.45 | 1.17 | 2.08 | 2.73 | FZR1 |
| 209416_s_at | 1.47 | 1.31 | 2.00 | 2.02 | FZR1 |
| 207574_s_at | 3.88 | 3.79 | 2.80 | 1.45 | GADD45B |
| 209304_x_at | 3.37 | 3.21 | 2.45 | 1.30 | GADD45B |
| 209305_s_at | 3.38 | 3.06 | 2.58 | 1.11 | GADD45B |
| 215248_at | 1.16 | 1.21 | 2.99 | 7.32 | GRB10 |
| 214438_at | 1.17 | 2.07 | 1.55 | 4.48 | HLX |
| 203665_at | 3.05 | 1.29 | 6.19 | 1.70 | HMOX1 |
| 204111_at | 1.02 | 0.65 | 0.30 | 0.19 | HNMT |
| 205580_s_at | 2.08 | 1.14 | 2.06 | 1.47 | HRH1 |
| 208937_s_at | 1.04 | 0.54 | 0.38 | 0.34 | ID1 |
| 206924_at | 9.37 | 2.46 | 4.91 | 2.70 | IL11 |
| 207952_at | 1.04 | 0.65 | 0.66 | 0.12 | IL5 |
| 204686_at | 0.41 | 0.80 | 0.67 | 0.50 | IRS1 |
| 209098_s_at | 2.26 | 2.06 | 1.76 | 2.45 | JAG1 |
| 41387_r_at | 1.22 | 1.64 | 2.02 | 2.06 | JMJD3 |
| 201464_x_at | 2.19 | 2.15 | 2.62 | 1.82 | JUN |
| 201466_s_at | 2.81 | 2.58 | 5.10 | 2.65 | JUN |
| 201473_at | 7.33 | 2.65 | 8.17 | 1.87 | JUNB |
| 218651_s_at | 2.19 | 3.08 | 3.96 | 2.86 | LARP6 |
| 221011_s_at | 2.32 | 1.87 | 5.40 | 6.95 | LBH |
| 218604_at | 1.62 | 2.08 | 2.67 | 1.23 | LEMD3 |
| 218574_s_at | 3.41 | 1.13 | 5.25 | 1.67 | LMCD1 |
| 204089_x_at | 1.40 | 1.56 | 2.18 | 2.04 | MAP3K4 |
| 208210_at | 1.00 | 0.69 | 0.21 | 0.41 | MAS1 |
| 214696_at | 2.02 | 1.46 | 2.26 | 1.51 | MGC14376 |
| 202519_at | 2.05 | 1.67 | 1.35 | 2.54 | MLXIP |
| 216303_s_at | 1.02 | 0.58 | 0.52 | 0.34 | MTMR1 |
| 213906_at | 0.41 | 0.85 | 0.83 | 0.42 | MYBL1 |
| 202431_s_at | 1.02 | 0.40 | 0.39 | 0.37 | MYC |
| 201496_x_at | 1.06 | 0.66 | 0.11 | 0.31 | MYH11 |
| 218149_s_at | 0.49 | 0.84 | 0.72 | 0.30 | NA |
| 207760_s_at | 2.50 | 1.11 | 2.43 | 1.91 | NCOR2 |
| 202607_at | 1.35 | 1.11 | 4.21 | 2.02 | NDST1 |
| 202150_s_at | 2.74 | 3.08 | 3.05 | 3.56 | NEDD9 |
| 201695_s_at | 2.12 | 1.26 | 1.14 | 2.08 | NP |
| 206699_x_at | 1.00 | 1.43 | 2.09 | 22.54 | NPAS1 |
| 209120_at | 0.19 | 0.64 | 0.49 | 0.34 | NR2F2 |
| 215073_s_at | 0.32 | 0.60 | 0.32 | 0.40 | NR2F2 |
| 213824_at | 0.91 | 0.71 | 0.50 | 0.23 | OLIG2 |
| 221868_at | 1.08 | 0.61 | 0.32 | 0.18 | PAIP2B |
| 206594_at | 1.05 | 1.80 | 3.47 | 2.08 | PASK |
| 221918_at | 2.17 | 2.27 | 1.34 | 1.61 | PCTK2 |
| 205463_s_at | 2.30 | 1.53 | 7.00 | 1.73 | PDGFA |
| 218691_s_at | 2.43 | 1.50 | 18.06 | 1.15 | PDLIM4 |
| 202464_s_at | 2.02 | 2.15 | 1.83 | 1.65 | PFKFB3 |
| 212134_at | 2.06 | 1.36 | 2.85 | 1.96 | PHLDB1 |
| 204612_at | 1.31 | 2.09 | 1.15 | 8.96 | PKIA |
| 204958_at | 1.20 | 1.48 | 6.24 | 2.08 | PLK3 |
| 209740_s_at | 0.87 | 0.83 | 0.49 | 0.24 | PNPLA4 |
| 218849_s_at | 2.22 | 1.41 | 2.83 | 1.57 | PPP1R13L |
| 202879_s_at | 1.29 | 2.10 | 1.42 | 2.16 | PSCD1 |
| 202880_s_at | 1.22 | 2.09 | 2.08 | 2.09 | PSCD1 |
| 206977_at | 0.96 | 1.23 | 15.95 | 4.12 | PTH |
| 219812_at | 1.18 | 1.19 | 2.43 | 2.52 | PVRIG |
| 216719_s_at | 1.06 | 0.50 | 0.16 | 0.93 | RAB11FIP4 |
| 219440_at | 1.10 | 0.79 | 0.21 | 0.16 | RAI2 |
| 203749_s_at | 3.39 | 1.93 | 4.10 | 1.46 | RARA |
| 206850_at | 1.23 | 1.34 | 4.49 | 2.30 | RASL10A |
| 203748_x_at | 1.42 | 1.33 | 2.02 | 2.21 | RBMS1 |
| 207266_x_at | 1.42 | 1.36 | 2.18 | 2.09 | RBMS1 |
| 209868_s_at | 1.42 | 1.37 | 2.07 | 2.06 | RBMS1 |
| 212099_at | 3.92 | 2.05 | 4.73 | 3.16 | RHOB |
| 205158_at | 0.71 | 0.78 | 0.48 | 0.26 | RNASE4 |
| 202627_s_at | 3.67 | 11.31 | 7.36 | 7.64 | SERPINE1 |
| 202656_s_at | 0.20 | 0.77 | 0.56 | 0.45 | SERTAD2 |
| 202657_s_at | 0.23 | 0.59 | 0.38 | 0.53 | SERTAD2 |
| 201739_at | 3.23 | 2.29 | 4.88 | 1.11 | SGK |
| 206675_s_at | 1.26 | 4.80 | 8.33 | 16.58 | SKIL |
| 217591_at | 2.02 | 3.25 | 3.13 | 2.52 | SKIL |
| 217685_at | 0.96 | 0.76 | 0.44 | 0.11 | SLC16A3 |
| 207298_at | 1.23 | 1.19 | 3.02 | 2.01 | SLC17A3 |
| 204790_at | 4.17 | 5.95 | 10.80 | 6.39 | SMAD7 |
| 210357_s_at | 1.60 | 2.29 | 2.40 | 3.03 | SMOX |
| 207390_s_at | 1.40 | 1.36 | 2.01 | 3.00 | SMTN |
| 212666_at | 1.62 | 1.66 | 2.30 | 2.02 | SMURF1 |
| 212668_at | 1.32 | 2.10 | 1.24 | 4.46 | SMURF1 |
| 215458_s_at | 1.49 | 1.31 | 2.00 | 2.36 | SMURF1 |
| 219480_at | 2.01 | 1.38 | 6.11 | 0.90 | SNAI1 |
| 219257_s_at | 2.36 | 1.66 | 2.11 | 2.09 | SPHK1 |
| 209875_s_at | 1.00 | 0.90 | 0.15 | 0.23 | SPP1 |
| 219677_at | 2.15 | 1.26 | 1.27 | 2.11 | SPSB1 |
| 217991_x_at | 1.43 | 1.10 | 2.43 | 2.29 | SSBP3 |
| 216917_s_at | 1.05 | 0.50 | 0.72 | 0.20 | SYCP1 |
| 212796_s_at | 1.73 | 1.29 | 1.81 | 2.26 | TBC1D2B |
| 211462_s_at | 1.04 | 1.24 | 8.78 | 2.08 | TBL1Y |
| 208398_s_at | 2.09 | 1.40 | 1.33 | 2.17 | TBPL1 |
| 221866_at | 1.47 | 1.35 | 5.80 | 2.91 | TFEB |
| 50221_at | 1.82 | 2.25 | 4.16 | 2.41 | TFEB |
| 211154_at | 0.99 | 0.49 | 0.80 | 0.08 | THPO |
| 217875_s_at | 5.44 | 2.96 | 31.67 | 1.62 | TMEPAI |
| 208296_x_at | 2.11 | 1.33 | 1.24 | 2.01 | TNFAIP8 |
| 218368_s_at | 1.36 | 2.00 | 2.11 | 1.39 | TNFRSF12A |
| 210987_x_at | 2.00 | 1.27 | 2.11 | 1.43 | TPM1 |
| 212664_at | 1.08 | 1.66 | 2.37 | 3.15 | TUBB4 |
| 205807_s_at | 2.92 | 1.85 | 2.23 | 2.75 | TUFT1 |
| 221098_x_at | 1.03 | 0.50 | 0.73 | 0.06 | UTP14A |
| 211527_x_at | 2.56 | 1.45 | 8.61 | 21.20 | VEGFA |
| 221423_s_at | 2.00 | 1.11 | 1.19 | 2.16 | YIPF5 |
| 208078_s_at | 3.17 | 2.49 | 2.47 | 2.05 | ZEB1 |
| 211962_s_at | 2.07 | 2.07 | 1.44 | 1.14 | ZFP36L1 |
| 211965_at | 1.56 | 2.28 | 1.43 | 3.34 | ZFP36L1 |
| 203520_s_at | 0.81 | 0.82 | 0.50 | 0.16 | ZNF318 |
| 221123_x_at | 0.42 | 0.96 | 0.93 | 0.50 | ZNF395 |
| 206810_at | 1.02 | 0.56 | 0.23 | 0.25 | ZNF44 |

TABLE 3
a 55 probe, unique 50 gene signature of all the genes that have univariate
associations with lung metastases. In ER− tumors: p = 0.0003 (originally 0.0003); In
LMS+ tumors: p = 0.000012 (originally < 0.0001)

| Probes | Direction | Corr.w.tgfb.act | Univariate.c | Univariate.p | Symbol |
|---|---|---|---|---|---|
| 203665_at | 1 | 0.329058167 | 0.6606244 | 2.16544E−07 | HMOX1 |
| 211527_x_at | 1 | 0.675990296 | 0.391253582 | 4.48942E−05 | VEGFA |
| 202627_s_at | 1 | 0.618184128 | 0.583283049 | 5.72343E−05 | SERPINE1 |
| 218818_at | 1 | 0.198070604 | 1.861044172 | 7.42988E−05 | FHL3 |
| 218618_s_at | 1 | 0.588177262 | 0.941372511 | 8.04662E−05 | FNDC3B |
| 211981_at | 1 | 0.65332303 | 0.76720787 | 9.15415E−05 | COL4A1 |
| 221423_s_at | 1 | 0.779285281 | 1.003581089 | 0.000104407 | YIPF5 |
| 221009_s_at | 1 | 0.167244747 | 0.699276019 | 0.000125438 | ANGPTL4 |
| 218368_s_at | 1 | 0.745860924 | 0.73385478 | 0.00014557 | TNFRSF12A |
| 209101_at | 1 | 0.465010719 | 0.687912875 | 0.000317061 | CTGF |
| 201169_s_at | 1 | 0.781081029 | 0.507509886 | 0.000545689 | BHLHB2 |
| 201464_x_at | 1 | 0.117512488 | 0.8944621 | 0.000792739 | JUN |
| 201329_s_at | 1 | 0.395700392 | 0.909128827 | 0.000854726 | ETS2 |
| 219774_at | 1 | 0.097559726 | 1.506265364 | 0.000898283 | CCDC93 |
| 211964_at | 1 | 0.280608916 | 0.941178307 | 0.001090799 | COL4A2 |
| 215458_s_at | 1 | 0.390897808 | 1.093314224 | 0.001102154 | SMURF1 |
| 219677_at | 1 | 0.243015361 | 1.129040601 | 0.001353386 | SPSB1 |
| 209868_s_at | 1 | 0.358172959 | 0.697085183 | 0.001534684 | RBMS1 |
| 207390_s_at | 1 | 0.621201404 | 0.805769931 | 0.00163468 | SMTN |
| 209098_s_at | 1 | 0.629640726 | 0.647901004 | 0.001856884 | JAG1 |
| 202519_at | 1 | 0.090323369 | 1.026190707 | 0.003185206 | MLXIP |
| 202150_s_at | 1 | 0.627467963 | 0.900781359 | 0.003297804 | NEDD9 |
| 221868_at | −1 | −0.287063403 | −0.561604122 | 0.003443685 | PAIP2B |
| 219440_at | −1 | −0.420481258 | −0.45681289 | 0.003543637 | RAI2 |
| 211980_at | 1 | 0.115960349 | 0.942133326 | 0.004472386 | COL4A1 |
| 209305_s_at | 1 | 0.610149662 | 0.621448467 | 0.00745052 | GADD45B |
| 201695_s_at | 1 | 0.168137981 | 0.755554958 | 0.016916518 | NP |
| 203748_x_at | 1 | 0.004854785 | 0.620751896 | 0.019243033 | RBMS1 |
| 221008_s_at | −1 | −0.248892145 | −0.447909816 | 0.020871059 | AGXT2L1 |
| 204174_at | 1 | 0.182789341 | 0.542718952 | 0.022888449 | ALOX5AP |
| 207266_x_at | 1 | 0.034497397 | 0.597727868 | 0.023298492 | RBMS1 |
| 212923_s_at | 1 | 0.054488441 | 0.824774431 | 0.023791129 | C6orf145 |
| 220842_at | −1 | −0.016216259 | −0.331869172 | 0.026528843 | AHI1 |
| 210987_x_at | 1 | 0.203854585 | 0.726674532 | 0.028998356 | TPM1 |
| 217591_at | 1 | 0.541379915 | 0.603351772 | 0.029257568 | SKIL |
| 201170_s_at | 1 | 0.09163886 | 0.644830027 | 0.029455813 | BHLHB2 |
| 210357_s_at | 1 | 0.058575277 | 0.504354773 | 0.032482652 | SMOX |
| 201473_at | 1 | 0.519203348 | 0.601295839 | 0.051762653 | JUNB |
| 217991_x_at | 1 | 0.32260368 | 0.505538738 | 0.062427707 | SSBP3 |
| 211962_s_at | 1 | 0.303134657 | 0.725288824 | 0.06986755 | ZFP36L1 |
| 206127_at | 1 | 0.463276453 | 0.532524896 | 0.076954196 | ELK3 |
| 219480_at | 1 | 0.302415429 | 0.371724664 | 0.080873546 | SNAI1 |
| 215252_at | 1 | 0.404833421 | 0.392246676 | 0.08527437 | DNAJC7 |
| 218149_s_at | −1 | −0.443065001 | −0.340459435 | 0.14103894 | NA |
| 206170_at | −1 | −0.500144981 | −0.340920044 | 0.154153385 | ADRB2 |
| 209740_s_at | −1 | −0.348644355 | −0.185608897 | 0.159872822 | PNPLA4 |
| 209416_s_at | 1 | 0.415951285 | 0.546068301 | 0.225087286 | FZR1 |
| 211965_at | 1 | 0.473642608 | 0.217399546 | 0.240922963 | ZFP36L1 |
| 217630_at | −1 | −0.316244317 | −0.140738019 | 0.480270579 | ANGEL2 |
| 209920_at | 1 | 0.458165297 | 0.116299309 | 0.503542922 | BMPR2 |
| 209120_at | −1 | −0.313588976 | −0.148157538 | 0.528961317 | NR2F2 |
| 202879_s_at | 1 | 0.375295213 | 0.070697027 | 0.561610299 | PSCD1 |
| 204686_at | −1 | −0.30793376 | −0.084058526 | 0.62988936 | IRS1 |
| 203973_s_at | −1 | −0.31970473 | −0.034431845 | 0.861159443 | CEBPD |
| 219427_at | −1 | −0.300823848 | −0.037838178 | 0.866880412 | FAT4 |

TABLE 4
20 gene signature. p = 0.0004, when applied to ER− tumors.

| Probes | Direction | Corr.w.tgfb.act | Univariate.c | Univariate.p | Symbol |
|---|---|---|---|---|---|
| 203665_at | 1 | 0.329058167 | 0.6606244 | 2.16544E−07 | HMOX1 |
| 211527_x_at | 1 | 0.675990296 | 0.391253582 | 4.48942E−05 | VEGFA |
| 202627_s_at | 1 | 0.618184128 | 0.583283049 | 5.72343E−05 | SERPINE1 |
| 218818_at | 1 | 0.198070604 | 1.861044172 | 7.42988E−05 | FHL3 |
| 218618_s_at | 1 | 0.588177262 | 0.941372511 | 8.04662E−05 | FNDC3B |
| 211981_at | 1 | 0.65332303 | 0.76720787 | 9.15415E−05 | COL4A1 |
| 221423_s_at | 1 | 0.779285281 | 1.003581089 | 0.000104407 | YIPF5 |
| 221009_s_at | 1 | 0.167244747 | 0.699276019 | 0.000125438 | ANGPTL4 |
| 218368_s_at | 1 | 0.745860924 | 0.73385478 | 0.00014557 | TNFRSF12A |
| 209101_at | 1 | 0.465010719 | 0.687912875 | 0.000317061 | CTGF |
| 201169_s_at | 1 | 0.781081029 | 0.507509886 | 0.000545689 | BHLHB2 |
| 201464_x_at | 1 | 0.117512488 | 0.8944621 | 0.000792739 | JUN |
| 201329_s_at | 1 | 0.395700392 | 0.909128827 | 0.000854726 | ETS2 |
| 219774_at | 1 | 0.097559726 | 1.506265364 | 0.000898283 | CCDC93 |
| 211964_at | 1 | 0.280608916 | 0.941178307 | 0.001090799 | COL4A2 |
| 215458_s_at | 1 | 0.390897808 | 1.093314224 | 0.001102154 | SMURF1 |
| 219677_at | 1 | 0.243015361 | 1.129040601 | 0.001353386 | SPSB1 |
| 209868_s_at | 1 | 0.358172959 | 0.697085183 | 0.001534684 | RBMS1 |
| 207390_s_at | 1 | 0.621201404 | 0.805769931 | 0.00163468 | SMTN |
| 209098_s_at | 1 | 0.629640726 | 0.647901004 | 0.001856884 | JAG1 |

TABLE 5
Different 20 gene signatures that are able to predict TGFβ responsiveness with
the associated p-values.

| Gene | Run 1 | Run 2 | Run 3 | Run 4 | Run 5 |
|---|---|---|---|---|---|
| Symbols | “FHL3 | “AHI1” | “IRS1” | “C6orf145” | “CTGF” |
| | “GADD45B | “C6orf145” | “PSCD1” | “AGXT2L1” | “GADD45B” |
| | “TNFRSF12A | “IRS1” | “TPM1” | “ADRB2” | “BHLHB2” |
| | “ETS2 | “COL4A1” | “C6orf145” | “PNPLA4” | “SERPINE1” |
| | “JAG1 | “JAG1” | “NEDD9” | “SERPINE1 | “NP” |
| | “SMURF1 | “SSBP3” | “HMOX1” | “ZFP36L1” | “ZNF395” |
| | “FAT4 | “ZFP36L1” | “RAI2” | “SKIL” | “MLXIP” |
| | “NR2F2 | “BHLHB2” | “NR2F2” | “JAG1” | “DNAJC7” |
| | “TPM1 | “ADRB2” | “FAT4” | “CCDC93” | “AGXT2L1” |
| | “JUN | “RAI2” | “SMTN” | “BMPR2” | “RBMS1” |
| | “CCDC93 | “AGXT2L1” | “ZFP36L1” | “ZFP36L1” | “TNFRSF12A |
| | “HMOX1 | “SPSB1 | “ALOX5AP | “FAT4” | “CCDC93” |
| | “RBMS1 | “ALOX5AP | “RBMS1” | “JUN” | “VEGFA” |
| | “BHLHB2 | “COL4A1 | “SMOX” | “ALOX5AP | “NR2F2” |
| | “ZFP36L1 | “GADD45B | “ANGPTL4” | “FZR1” | “COL4A1” |
| | “SSBP3 | “SMURF1” | “PAIP2B” | “NR2F2” | “FNDC3B” |
| | “ZNF395 | “BHLHB2” | “BHLHB2” | “FHL3” | “SPSB1” |
| | “SKIL” | “JUNB” | “RBMS1” | “COL4A1” | “PNPLA4” |
| | “FZR1” | “SMTN” | “JUN” | “CEBPD” | “PAIP2B” |
| | “RAI2” | “NEDD9” | “COL4A1” | “RAI2” | “BMPR2” |
| p values | 0.006 | 4.96E+00 | 9.61E−05 | 0.002 | 3.32E−05 |

TABLE 6
TBRS status was assessed in each of the
breast cancer metastasis samples submitted for microarray analysis.
The percentage of TBRS positive samples in each
metastasis site is shown.

| Site | TBRS+ (%) |
|---|---|
| Lung | 14/18 (78) |
| Bone | 8/16 (50) |
| Brain | 6/19 (32) |
| Liver | 5/5 (100) |
| Other sites* | 5/9 (56) |
| All sites | 38/67 (58) |

*Includes ovary, duodenum and chest wall

TABLE 7
Univariate and multivariate analyses correlating TGFβ pathway
components and incidence of lung metastasis in the breast cancer
patient cohorts. P-values for these associations are listed.

| | All tumors | ER− tumors |
|---|---|---|
| TGFBR1 | 0.7740 | 0.2560 |
| TGFBR2 | 0.1700 | 0.0110 |
| TGFB1 | 0.5460 | 0.1800 |
| TGFB2 | 0.8750 | 0.5120 |
| TGFB3 | 0.001^{a} | 0.0271^{a} |
| Smad2 | 0.1990 | 0.0425 |
| Smad3 | 0.3090 | 0.0111 |
| Smad4 | 0.0937 | 0.1780 |
| TGFBR2 + Smad3^{b} | 0.3380 | 0.0106 |
| All (except TGFB3)^{b} | 0.6260 | 0.0533 |
| TBRS | 0.0133 | <0.0001 |

^{a}Negative correlation
^{b}Multivariate analyses using Cox proportional hazards regression model

### REFERENCES

Akhurst, R. J., and Derynck, R. (2001). TGF-beta signaling in cancer—a double-edged sword. Trends Cell Biol 11, S44-51.
Backhed, F., Crawford, P. A., O'Donnell, D., and Gordon, J. I. (2007). Postnatal lymphatic partitioning from the blood vasculature in the small intestine requires fasting-induced adipose factor. Proc Natl Acad Sci USA 104, 606-611.
Bernards, R., and Weinberg, R. A. (2002). A progression puzzle. Nature 418, 823.
Bhowmick, N. A., Chytil, A., Plieth, D., Gorska, A. E., Dumont, N., Shappell, S., Washington, M. K., Neilson, E. G., and Moses, H. L. (2004). TGF-beta signaling in fibroblasts modulates the oncogenic potential of adjacent epithelia. Science 303, 848-851.
Bierie, B., and Moses, H. L. (2006). Tumour microenvironment: TGFbeta: the molecular Jekyll and Hyde of cancer. Nat Rev Cancer 6, 506-520.
Bild, A. H., Potti, A., and Nevins, J. R. (2006). Linking oncogenic pathways with therapeutic opportunities. Nat Rev Cancer 6, 735-741.
Brummelkamp, T. R., Bernards, R., and Agami, R. (2002). Stable suppression of tumorigenicity by virus-mediated RNA interference. Cancer Cell 2, 243-247.
Buck, M. B., Fritz, P., Dippon, J., Zugmaier, G., and Knabbe, C. (2004). Prognostic significance of transforming growth factor beta receptor II in estrogen receptor-negative breast cancer patients. Clin Cancer Res 10, 491-498.
Cailleau, R., Olive, M., and Cruciger, Q. V. (1978). Long-term human breast carcinoma cell lines of metastatic origin: preliminary characterization. In Vitro 14, 911-915.
Calonge, M. J., and Massagué, J. (1999). Smad4/DPC4 silencing and hyperactive Ras jointly disrupt transforming growth factor-beta antiproliferative responses in colon cancer cells. J Biol Chem 274, 33637-33643.
Camenisch, G., Pisabarro, M. T., Sherman, D., Kowalski, J., Nagel, M., Hass, P., Xie, M. H., Gurney, A., Bodary, S., Liang, X. H., et al. (2002). ANGPTL3 stimulates endothelial cell adhesion and migration via integrin alpha vbeta 3 and induces blood vessel formation in vivo. J Biol Chem 277, 17281-17290.
Cazes, A., Galaup, A., Chomel, C., Bignon, M., Brechot, N., Le Jan, S., Weber, H.,
Corvol, P., Muller, L., Germain, S., et al. (2006). Extracellular matrix-bound angiopoietin-like 4 inhibits endothelial cell adhesion, migration, and sprouting and alters actin cytoskeleton. Circ Res 99, 1207-1215.
Chang, H. Y., Nuyten, D. S., Sneddon, J. B., Hastie, T., Tibshirani, R., Sorlie, T., Dai, H., He, Y. D., van't Veer, L. J., Bartelink, H., et al. (2005). Robustness, scalability, and integration of a wound-response gene expression signature in predicting breast cancer survival. Proc Natl Acad Sci USA 102, 3738-3743.
Chen, C. R., Kang, Y., and Massagué, J. (2001). Defective repression of c-myc in breast cancer cells: A loss at the core of the transforming growth factor beta growth arrest program. Proc Natl Acad Sci USA 98, 992-999.
Chen, C. R., Kang, Y., Siegel, P. M., and Massagué, J. (2002). E2F4/5 and p107 as Smad cofactors linking the TGFbeta receptor to c-myc repression. Cell 110, 19-32.
Chen, T., Carter, D., Garrigue-Antar, L., and Reiss, M. (1998). Transforming growth factor beta type I receptor kinase mutant associated with metastatic breast cancer. Cancer Res 58, 4805-4810.
Dalal, B. I., Keown, P. A., and Greenberg, A. H. (1993). Immunocytochemical localization of secreted transforming growth factor-beta 1 to the advancing edges of primary tumors and to lymph node metastases of human mammary carcinoma. Am J Pathol 143, 381-389. Dejana, E. (2004). Endothelial cell-cell junctions: happy together. Nat Rev Mol Cell Biol 5, 261-270.
Desai, U., Lee, E. C., Chung, K., Gao, C., Gay, J., Key, B., Hansen, G., Machajewski, D., Platt, K. A., Sands, A. T., et al. (2007). Lipid-lowering effects of anti-angiopoietin-like 4 antibody recapitulate the lipid phenotype found in angiopoietin-like 4 knockout mice. Proc Natl Acad Sci USA 104, 11766-11771.
Dumont, N., and Arteaga, C. L. (2003). Targeting the TGF beta signaling network in human neoplasia. Cancer Cell 3, 531-536.
Fidler, I. J. (2003). The pathogenesis of cancer metastasis: the ‘seed and soil’ hypothesis revisited. Nat Rev Cancer 3, 453-458.
Forrester, E., Chytil, A., Bierie, B., Aakre, M., Gorska, A. E., Sharif-Afshar, A. R., Muller, W. J., and Moses, H. L. (2005). Effect of conditional knockout of the type II TGF-beta receptor gene in mammary epithelia on mammary gland development and polyomavirus middle T antigen induced tumor formation and metastasis. Cancer Res 65, 2296-2302.
Galaup, A., Cazes, A., Le Jan, S., Philippe, J., Connault, E., Le Coz, E., Mekid, H., Mir, L. M., Opolon, P., Corvol, P., et al. (2006). Angiopoietin-like 4 prevents metastasis through inhibition of vascular permeability and tumor cell motility and invasiveness. Proc Natl Acad Sci USA 103, 18721-18726.
Gale, N. W., Thurston, G., Hackett, S. F., Renard, R., Wang, Q., McClain, J., Martin, C., Witte, C., Witte, M. H., Jackson, D., et al. (2002). Angiopoietin-2 is required for postnatal angiogenesis and lymphatic patterning, and only the latter role is rescued by Angiopoietin-1. Dev Cell 3, 411-423.
Gobbi, H., Arteaga, C. L., Jensen, R. A., Simpson, J. F., Dupont, W. D., Olson, S. J., Schuyler, P. A., Plummer, W. D., Jr., and Page, D. L. (2000). Loss of expression of transforming growth factor beta type II receptor correlates with high tumour grade in human breast in-situ and invasive carcinomas. Histopathology 36, 168-177.
Gomis, R. R., Alarcon, C., Nadal, C., Van Poznak, C., and Massagué, J. (2006). C/EBPbeta at the core of the TGFbeta cytostatic response and its evasion in metastatic breast cancer cells. Cancer Cell 10, 203-214.
Gorelik, L., and Flavell, R. A. (2000). Abrogation of TGFbeta signaling in T cells leads to spontaneous T cell differentiation and autoimmune disease. Immunity 12, 171-181.
Gupta, G. P., Nguyen, D. X., Chiang, A. C., Bos, P. D., Kim, J. Y., Nadal, C., Gomis, R. R., Manova-Todorova, K., and Massagué, J. (2007a). Mediators of vascular remodelling co-opted for sequential steps in lung metastasis. Nature 446, 765-770.
Gupta, G. P., Perk, J., Acharyya, S., de Candia, P., Mittal, V., Todorova-Manova, K., Gerald, W. L., Brogi, E., Benezra, R., and Massagué, J. (2007b). ID genes mediate tumor reinitiation during breast cancer lung metastasis. Proc Natl Acad Sci USA.
Gupta, G. P. a. M., J. (2006). Cancer metastasis: building a framework. Cell 127, 679-695.
Hermann, L. M., Pinkerton, M., Jennings, K., Yang, L., Grom, A., Sowders, D., Kersten, S., Witte, D. P., Hirsch, R., and Thornton, S. (2005). Angiopoietin-like-4 is a potential angiogenic mediator in arthritis. Clin Immunol 115, 93-101.
Hynes, R. O. (2003). Metastatic potential: generic predisposition of the primary tumor or rare, metastatic variants- or both? Cell 113, 821-823.
Ito, Y., Oike, Y., Yasunaga, K., Hamada, K., Miyata, K., Matsumoto, S., Sugano, S., Tanihara, H., Masuho, Y., and Suda, T. (2003). Inhibition of angiogenesis and vascular leakiness by angiopoietin-related protein 4. Cancer Res 63, 6651-6657.
Kang, Y., Chen, C. R., and Massagué, J. (2003a). A self-enabling TGFbeta response coupled to stress signaling: Smad engages stress response factor ATF3 for Id1 repression in epithelial cells. Mol Cell 11, 915-926.
Kang, Y., He, W., Tulley, S., Gupta, G. P., Serganova, I., Chen, C. R., Manova-Todorova, K., Blasberg, R., Gerald, W. L., and Massagué, J. (2005). Breast cancer bone metastasis mediated by the Smad tumor suppressor pathway. Proc Natl Acad Sci USA 102, 13909-13914.
Kang, Y., Siegel, P. M., Shu, W., Drobnjak, M., Kakonen, S. M., Cordon-Cardo, C., Guise, T. A., and Massagué, J. (2003b). A multigenic program mediating breast cancer metastasis to bone. Cancer Cell 3, 537-549.
Kaplan, R. N., Riba, R. D., Zacharoulis, S., Bramley, A. H., Vincent, L., Costa, C., MacDonald, D. D., Jin, D. K., Shido, K., Kerns, S. A., et al. (2005). VEGFR1-positive haematopoietic bone marrow progenitors initiate the pre-metastatic niche. Nature 438, 820-827.
Kersten, S., Mandard, S., Tan, N. S., Escher, P., Metzger, D., Chambon, P., Gonzalez, F. J., Desvergne, B., and Wahli, W. (2000). Characterization of the fasting-induced adipose factor FIAF, a novel peroxisome proliferator-activated receptor target gene. J Biol Chem 275, 28488-28493.
Kielhorn, E., Schofield, K., and Rimm, D. L. (2002). Use of magnetic enrichment for detection of carcinoma cells in fluid specimens. Cancer 94, 205-211.
Kim, I., Kim, H. G., Kim, H., Kim, H. H., Park, S. K., Uhm, C. S., Lee, Z. H., and Koh, G. Y. (2000). Hepatic expression, synthesis and secretion of a novel fibrinogen/angiopoietin-related protein that prevents endothelial-cell apoptosis. Biochem J 346 Pt 3, 603-610.
Kim, M., Gans, J. D., Nogueira, C., Wang, A., Paik, J. H., Feng, B., Brennan, C., Hahn, W. C., Cordon-Cardo, C., Wagner, S. N., et al. (2006). Comparative oncogenomics identifies NEDD9 as a melanoma metastasis gene. Cell 125, 1269-1281.
Laping, N. J., Grygielko, E., Mathur, A., Butter, S., Bomberger, J., Tweed, C., Martin, W., Formwald, J., Lehr, R., Harling, J., et al. (2002). Inhibition of transforming growth factor (TGF)-beta1-induced extracellular matrix with a novel inhibitor of the TGF-beta type I receptor kinase activity: SB-431542. Mol Pharmacol 62, 58-64.
Le Jan, S., Amy, C., Gazes, A., Monnot, C., Lamande, N., Favier, J., Philippe, J., Sibony, M., Gasc, J. M., Corvol, P., et al. (2003). Angiopoietin-like 4 is a proangiogenic factor produced during ischemia and in conventional renal cell carcinoma. Am J Pathol 162, 1521-1528.
Levy, L., and Hill, C. S. (2006). Alterations in components of the TGF-beta superfamily signaling pathways in human cancer. Cytokine Growth Factor Rev 17, 41-58.
Lucke, C. D., Philpott, A., Metcalfe, J. C., Thompson, A. M., Hughes-Davies, L., Kemp, P. R., and Hesketh, R. (2001). Inhibiting mutations in the transforming growth factor beta type 2 receptor in recurrent human breast cancer. Cancer Res 61, 482-485.
Lynch, C. C., Hikosaka, A., Acuff, H. B., Martin, M. D., Kawai, N., Singh, R. K., Vargo-Gogola, T. C., Begtrup, J. L., Peterson, T. E., Fingleton, B., et al. (2005). MMP-7 promotes prostate cancer-induced osteolysis via the solubilization of RANKL. Cancer Cell 7, 485-496.
Massagué, J., Blain, S. W., and Lo, R. S. (2000). TGFbeta signaling in growth control, cancer, and heritable disorders. Cell 103, 295-309.
Massagué, J., and Gomis, R. R. (2006). The logic of TGFbeta signaling. FEBS Lett 580, 2811-2820.
Massagué, J., Seoane, J., and Wotton, D. (2005). Smad transcription factors. Genes Dev 19, 2783-2810.
Minn, A. J., Gupta, G. P., Padua, D., Bos, P., Nguyen, D. X., Nuyten, D., Kreike, B., Zhang, Y., Wang, Y., Ishwaran, H., et al. (2007). Lung metastasis genes couple breast tumor size and metastatic spread. Proc Natl Acad Sci USA 104, 6740-6745.
Minn, A. J., Gupta, G. P., Siegel, P. M., Bos, P. D., Shu, W., Giri, D. D., Viale, A., Olshen, A. B., Gerald, W. L., and Massagué, J. (2005). Genes that mediate breast cancer metastasis to lung. Nature 436, 518-524.
Mundy, G. R. (2002). Metastasis to bone: causes, consequences and therapeutic opportunities. Nat Rev Cancer 2, 584-593.
Muraoka, R. S., Dumont, N., Ritter, C. A., Dugger, T. C., Brantley, D. M., Chen, J., Easterly, E., Roebuck, L. R., Ryan, S., Gotwals, P. J., et al. (2002). Blockade of TGF-beta inhibits mammary tumor cell viability, migration, and metastases. J Clin Invest 109, 1551-1559.
Muraoka, R. S., Koh, Y., Roebuck, L. R., Sanders, M. E., Brantley-Sieders, D., Gorska, A. E., Moses, H. L., and Arteaga, C. L. (2003). Increased malignancy of Neu-induced mammary tumors overexpressing active transforming growth factor beta 1. Mol Cell Biol 23, 8691-8703.
Nguyen, D. X., and Massagué, J. (2007). Genetic determinants of cancer metastasis. Nat Rev Genet 8, 341-352.
Oghiso, Y., and Matsuoka, O. (1979). Distribution of colloidal carbon in lymph nodes of mice injected by different routes. Jpn J Exp Med 49, 223-234.
Oike, Y., Yasunaga, K., and Suda, T. (2004). Angiopoietin-related/angiopoietin-like proteins regulate angiogenesis. Int J Hematol 80, 21-28.
Ory, D. S., Neugeboren, B. A., and Mulligan, R. C. (1996). A stable human-derived packaging cell line for production of high titer retrovirus/vesicular stomatitis virus G pseudotypes. Proc Natl Acad Sci USA 93, 11400-11406.
Paget, S. (1889). The distribution of secondary growths in cancer of the breast. Lancet 1, 571-573.
Padua, D. (2008). TGFβ primes breast tumors for lung metastasis seeding through angiopoietin-like 4. Cell 133(1), 66-77.
Parikh, S. M., Mammoto, T., Schultz, A., Yuan, H. T., Christiani, D., Karumanchi, S. A., and Sukhatme, V. P. (2006). Excess circulating angiopoietin-2 may contribute to pulmonary vascular leak in sepsis in humans. PLoS Med 3, e46.
Ponomarev, V., Doubrovin, M., Serganova, I., Vider, J., Shavrin, A., Beresten, T., Ivanova, A., Ageyeva, L., Tourkova, V., Balatoni, J., et al. (2004). A novel triple-modality reporter gene for whole-body fluorescent, bioluminescent, and nuclear noninvasive imaging. Eur J Nucl Med Mol Imaging 31, 740-751.
Seoane, J., Le, H. V., Shen, L., Anderson, S. A., and Massagué, J. (2004). Integration of Smad and forkhead pathways in the control of neuroepithelial and glioblastoma cell proliferation. Cell 117, 211-223.
McSherry E A, Donatello S, Hopkins A M, McDonnell S., (2007). Molecular basis of invasion in breast cancer. Cell Mol Life Sci, 64(24):3201-18
Shipitsin, M., Campbell, L. L., Argani, P., Weremowicz, S., Bloushtain-Qimron, N., Yao, J., Nikolskaya, T., Serebryiskaya, T., Beroukhim, R., Hu, M., et al. (2007). Molecular definition of breast tumor heterogeneity. Cancer Cell 11, 259-273.
Siegel, P. M., and Massagué, J. (2003). Cytostatic and apoptotic actions of TGF-beta in homeostasis and cancer. Nat Rev Cancer 3, 807-821.
Siegel, P. M., Shu, W., Cardiff, R. D., Muller, W. J., and Massagué, J. (2003). Transforming growth factor beta signaling impairs Neu-induced mammary tumorigenesis while promoting pulmonary metastasis. Proc Natl Acad Sci USA 100, 8430-8435.
Sorlie, T., Tibshirani, R., Parker, J., Hastie, T., Marron, J. S., Nobel, A., Deng, S., Johnsen, H., Pesich, R., Geisler, S., et al. (2003). Repeated observation of breast tumor subtypes in independent gene expression data sets. Proc Natl Acad Sci USA 100, 8418-8423.
Sporn, M. B., and Roberts, A. B. (1990). TGF-beta: problems and prospects. Cell Regul 1, 875-882.
Sukonina, V., Lookene, A., Olivecrona, T., and Olivecrona, G. (2006). Angiopoietin-like protein 4 converts lipoprotein lipase to inactive monomers and modulates lipase activity in adipose tissue. Proc Natl Acad Sci USA 103, 17450-17455.
Thiery, J. P. (2002). Epithelial-mesenchymal transitions in tumour progression. Nat Rev Cancer 2, 442-454.
Thomas, D. A., and Massagué, J. (2005). TGF-beta directly targets cytotoxic T cell functions during tumor evasion of immune surveillance. Cancer Cell 8, 369-380.
van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.
Wakefield, L. M., and Roberts, A. B. (2002). TGF-beta signaling: positive and negative effects on tumorigenesis. Curr Opin Genet Dev 12, 22-29.
Wang, Y., Klijn, J. G., Zhang, Y., Sieuwerts, A. M., Look, M. P., Yang, F., Talantov, D., Timmermans, M., Meijer-van Gelder, M. E., Yu, J., et al. (2005). Gene-expression profiles to predict distant metastasis of lymph-node-negative primary breast cancer. Lancet 365, 671-679.
Weis-Garcia, F., and Massagué, J. (1996). Complementation between kinase-defective and activation-defective TGF-beta receptors reveals a novel form of receptor cooperativity essential for signaling. Embo J 15, 276-289.
Welch, D. R., Fabra, A., and Nakajima, M. (1990). Transforming growth factor beta stimulates mammary adenocarcinoma cell invasion and metastatic potential. Proc Natl Acad Sci USA 87, 7678-7682.
Yin, J. J., Selander, K., Chirgwin, J. M., Dallas, M., Grubbs, B. G., Wieser, R., Massagué, J., Mundy, G. R., and Guise, T. A. (1999). TGF-beta signaling blockade inhibits PTHrP secretion by breast cancer cells and bone metastases development. J Clin Invest 103, 197-206.
Yoon, J. C., Chickering, T. W., Rosen, E. D., Dussault, B., Qin, Y., Soukas, A., Friedman, J. M., Holmes, W. E., and Spiegelman, B. M. (2000). Peroxisome proliferator-activated receptor gamma target gene encoding a novel angiopoietin-related protein associated with adipose differentiation. Mol Cell Biol 20, 5343-5349.

1. A method of diagnosing metastatic potential of cancer cells comprising
obtaining a diagnostic signature from cancer cells indicative of the metastatic potential of the cancer cells;
wherein said diagnostic signature is obtained by measuring levels in cancer cells from the patient of five or more markers selected from the group of genes typifying the TGFβ response in human epithelial cells;
comparing said diagnostic signature to a control signature; and
based on the comparison, giving a prognosis of a high risk for metastasis if the diagnostic signature is different from the control signature by at least a threshold amount.

2. The method of claim 1 wherein the group of genes typifying the TGFβ response in human epithelial cells from which the five or more markers are selected consists of ABTB2, ACSBG1, ADCK2, ADRB2, AGTR2, AGXT2L1, AHI1, ALOX5AP, AMIGO2, ANGEL2, ANGPTL4, ANK1, ARFGAP1, ARHGAP12, ARID5B, ARL6IP2, ATF7IP, AVPI1, BET1L, BHLHB2, BHMT, BMPR2, BRDT, C13 orf15, C18orf25, C3orf28, C3orf52, C6orf145, C6orf148, CCDC93, CD163, CD1E, CD28, CDKN1A, CDKN2AIP, CEBPD, CENPF, CITED2, CITED2, CKMT2, COL1A1, COL4A2, COL8A2, CTGF, CUBN, CYBB, DDIT4, DNAJC7, DOPEY1, EDN1, ELK3, ETS2, FAT4, FGB, FHL3, FILIP1L, FLJ10357, FLT4, FLVCR2, FNDC3B, FSTL3, FZR1, GADD45B, GRB10, HLX, HMOX1, HNMT, HRH1, ID1, IL11, IL5, IRS1, JAG1, JMJD3, JUN, JUNB, LARP6, LBH, LEMD3, LMCD1, MAP3K4, MAS1, MGC14376, MLXIP, MTMR1, MYBL1, MYC, MYH11, NA, NCOR2, NDST1, NEDD9, NP, NPAS1, NR2F2, OLIG2, PAIP2B, PASK, PCTK2, PDGFA, PDLIM4, PFKFB3, PHLDB1, PKIA, PLK3, PNPLA4, PPP1R13L, PSCD1, PSCD1, PTH, PVRIG, RAB11FIP4, RAI2, RARA, RASL10A, RBMS1, RHOB, RNASE4, SERPINE1, SERTAD2, SGK, SKIL, SLC16A3, SLC17A3, SMAD7, SMOX, SMTN, SMURF1, SNAI1, SPHK1, SPP1, SPSB1, SSBP3, SYCP1, TBC1D2B, TBL1Y, TBPL1, TFEB, THPO, TMEPAI, TNFAIP8, TNFRSF12A, TPM1, TUBB4, TUFT1, UTP14A, VEGFA, YIPF5, ZEB1, ZFP36L1, ZNF318, ZNF395, and ZNF44.

3. The method of claim 2 wherein the group of genes typifying the TGFβ response in human epithelial cells from which the five or more markers are selected consists of HMOX1, VEGFA, SERPINE1, FHL3, FNDC3B, COL4A1, YIPF5, ANGPTL4, TNFRSF12A, CTGF, JUN, ETS2, CCDC93, COL4A2, SMURF1, SPSB1, SMTN, JAG1, MLXIP, NEDD9, PAIP2B, RAI2, GADD45B, NP, AGXT2L1, ALOX5AP, RBMS1, C6orf145, AHI1, TPM1, SKIL, BHLHB2, SMOX, JUNB, SSBP3, ELK3, SNAI1, DNAJC7, NA, ADRB2, PNPLA4, FZR1, ZFP36L1, ANGEL2, BMPR2, NR2F2, PSCD1, IRS1, CEBPD, and FAT4.

4. The method of claim 2 wherein the group of genes typifying the TGFβ response in human epithelial cells from which the five or more markers are selected consists of BHLHB2, COL4A1, CCDC93, JAG1, JUN, NR2F2, RAI2, RBMS1, ZFP36L1, AGXT2L1, ALOX5AP, C6orf145, FAT4, FHL3, GADD45B, HMOX1, SERPINE1, SMTN, SMURF1, SPSB1, TNFRSF12A, ADRB2, ANGPTL4, BMPR2, CTGF, ETS2, FNDC3B, FZR1, IRS1, NEDD9, PAIP2B, PNPLA4, SKIL, SSBP3, TPM1, VEGFA, ZNF395, AHI1, CEBPD, COL4A2, DNAJC7, JUNB, MLXIP, NP, PSCD1, SMOX, and YIPF5.

5. The method of claim 4 wherein the group of genes typifying the TGFβ response in human epithelial cells from which the five or more markers are selected consists of HMOX1, VEGFA, SERPINE1, FHL3, FNDC3B, COL4A1, YIPF5, ANGPTL4, TNFRSF12A, CTGF, BHLHB2, JUN, ETS2, CCDC93, COL4A2, SMURF1, SPSB1, RBMS1, SMTN, and JAG1.

6. The method of claim 4 wherein the group of genes typifying the TGFβ response in human epithelial cells from which the five or more markers are selected consists of FHL3, GADD45B, TNFRSF12A, ETS2, JAG1, SMURF1, FAT4, NR2F2, TPM1, JUN, CCDC93, HMOX1, RBMS1, BHLHB2, ZFP36L1, SSBP3, ZNF395, SKIL, FZR1, and RAI2.

7. The method of claim 4 wherein the group of genes typifying the TGFβ response in human epithelial cells from which the five or more markers are selected consists of AHI1, C6orf145, IRS1, COL4A1, JAG1, SSBP3, ZFP36L1, ADRB2, RAI2, AGXT2L1, SPSB1, ALOX5AP, GADD45B, SMURF1, JUNB, SMTN, and NEDD9.

8. The method of claim 4 wherein the group of genes typifying the TGFβ response in human epithelial cells from which the five or more markers are selected consists of IRS1, PSCD1, TPM1, C6orf145, NEDD9, HMOX1, RAI2, NR2F2, FAT4, SMTN, ZFP36L1, ALOX5AP, RBMS1, SMOX, ANGPTL4, PAIP2B, BHLHB2, RBMS1, JUN, and COL4A1.

9. The method of claim 4 wherein the group of genes typifying the TGFβ response in human epithelial cells from which the five or more markers are selected consists of C6orf145, AGXT2L1, ADRB2, PNPLA4, SERPINE1, ZFP36L1, SKIL, JAG1, CCDC93, BMPR2, ZFP36L1, FAT4, JUN, ALOX5AP, FZR1, NR2F2, FHL3, COL4A1, CEBPD, and RAI2.

10. The method of claim 4 wherein the group of genes typifying the TGFβ response in human epithelial cells from which the five or more markers are selected consists of CTGF, GADD45B, BHLHB2, SERPINE1, NP, ZNF395, MLXIP, DNAJC7, AGXT2L1, RBMS1, TNFRSF12A, CCDC93, VEGFA, NR2F2, COL4A1, FNDC3B, SPSB1, PNPLA4, PAIP2B, and BMPR2.

11. The method of claim 1 further comprising determining whether the cancer cells are estrogen receptor negative; and
based on this determination, giving a prognosis of a high risk for metastasis if the cells are estrogen receptor negative and if the diagnostic signature is greater than the control signature by a threshold amount.

12. The method of claim 11 further comprising determining whether the cancer cells are LMS positive; and
based on this determination, giving a prognosis of a high risk for metastasis if the cells are estrogen receptor negative, and LMS positive, and if the diagnostic signature is greater than the control signature by a threshold amount.

13. The method of claim 1 further comprising determining whether the cancer cells are LMS positive; and
based on this determination, giving a prognosis of a high risk for metastasis if the cells are LMS positive, and if the diagnostic signature is greater than the control signature by a threshold amount.

14. The method of claim 1 wherein the cancer cells are breast cancer cells.

15. The method of claim 1 wherein the cancer cells are melanoma cells.

16. The method of any of the preceding claims wherein the group of genes consists of twenty genes.

17. A kit for diagnosing metastatic potential of cancer comprising reagents for determining the expression levels of a set five or more markers selected from the group of genes typifying the TGFβ response in human epithelial cells.

18. The kit of claim 17, wherein the group of genes typifying the TGFβ response in human epithelial cells from which the five or more markers are selected consists of ABTB2, ACSBG1, ADCK2, ADRB2, AGTR2, AGXT2L1, AHI1, ALOX5AP, AMIGO2, ANGEL2, ANGPTL4, ANK1, ARFGAP1, ARHGAP12, ARID5B, ARL6IP2, ATF7IP, AVPI1, BET1L, BHLHB2, BHMT, BMPR2, BRDT, C13orf15, C18orf25, C3orf28, C3orf52, C6orf145, C6 orf148, CCDC93, CD163, CD1E, CD28, CDKN1A, CDKN2AIP, CEBPD, CENPF, CITED2, CITED2, CKMT2, COL1A1, COL4A2, COL8A2, CTGF, CUBN, CYBB, DDIT4, DNAJC7, DOPEY1, EDN1, ELK3, ETS2, FAT4, FGB, FHL3, FILIP1L, FLJ10357, FLT4, FLVCR2, FNDC3B, FSTL3, FZR1, GADD45B, GRB10, HLX, HMOX1, HNMT, HRH1, ID1, IL11, IL5, IRS1, JAG1, JMJD3, JUN, JUNB, LARP6, LBH, LEMD3, LMCD1, MAP3K4, MAS1, MGC14376, MLXIP, MTMR1, MYBL1, MYC, MYH11, NA, NCOR2, NDST1, NEDD9, NP, NPAS1, NR2F2, OLIG2, PAIP2B, PASK, PCTK2, PDGFA, PDLIM4, PFKFB3, PHLDB1, PKIA, PLK3, PNPLA4, PPP1R13L, PSCD1, PSCD1, PTH, PVRIG, RAB11FIP4, RAI2, RARA, RASL10A, RBMS1, RHOB, RNASE4, SERPINE1, SERTAD2, SGK, SKIL, SLC16A3, SLC17A3, SMAD7, SMOX, SMTN, SMURF1, SNAI1, SPHK1, SPP1, SPSB1, SSBP3, SYCP1, TBC1D2B, TBL1Y, TBPL1, TFEB, THPO, TMEPAI, TNFAIP8, TNFRSF12A, TPM1, TUBB4, TUFT1, UTP14A, VEGFA, YIPF5, ZEB1, ZFP36L1, ZNF318, ZNF395, and ZNF44.

19. The kit of claim 17 wherein the group of genes typifying the TGFβ response in human epithelial cells from which the five or more markers are selected consists of:
(a) HMOX1, VEGFA, SERPINE1, FHL3, FNDC3B, COL4A1, YIPF5, ANGPTL4, TNFRSF12A, CTGF, JUN, ETS2, CCDC93, COL4A2, SMURF1, SPSB1, SMTN, JAG1, MLXIP, NEDD9, PAIP2B, RAI2, GADD45B, NP, AGXT2L1, ALOX5AP, RBMS1, C6orf145, AHI1, TPM1, SKIL, BHLHB2, SMOX, JUNB, SSBP3, ELK3, SNAI1, DNAJC7, NA, ADRB2, PNPLA4, FZR1, ZFP36L1, ANGEL2, BMPR2, NR2F2, PSCD1, IRS1, CEBPD, and FAT4,
(b) BHLHB2, COL4A1, CCDC93, JAG1, JUN, NR2F2, RAI2, RBMS1, ZFP36L1, AGXT2L1, ALOX5AP, C6orf145, FAT4, FHL3, GADD45B, HMOX1, SERPINE1, SMTN, SMURF1, SPSB1, TNFRSF12A, ADRB2, ANGPTL4, BMPR2, CTGF, ETS2, FNDC3B, FZR1, IRS1, NEDD9, PAIP2B, PNPLA4, SKIL, SSBP3, TPM1, VEGFA, ZNF395, AHI1, CEBPD, COL4A2, DNAJC7, JUNB, MLXIP, NP, PSCD1, SMOX, and YIPF5;
(c) HMOX1, VEGFA, SERPINE1, FHL3, FNDC3B, COL4A1, YIPF5, ANGPTL4, TNFRSF12A, CTGF, BHLHB2, JUN, ETS2, CCDC93, COL4A2, SMURF1, SPSB1, RBMS1, SMTN, and JAG1;
(d) FHL3, GADD45B, TNFRSF12A, ETS2, JAG1, SMURF1, FAT4, NR2F2, TPM1, JUN, CCDC93, HMOX1, RBMS1, BHLHB2, ZFP36L1, SSBP3, ZNF395, SKIL, FZR1, and RAI2;
(e) AHI1, C6orf145, IRS1, COL4A1, JAG1, SSBP3, ZFP36L1, ADRB2, RAI2, AGXT2L1, SPSB1, ALOX5AP, GADD45B, SMURF1, JUNB, SMTN, and NEDD9;
(f) IRS1, PSCD1, TPM1, C6orf145, NEDD9, HMOX1, RAI2, NR2F2, FAT4, SMTN, ZFP36L1, ALOX5AP, RBMS1, SMOX, ANGPTL4, PAIP2B, BHLHB2, RBMS1, JUN, and COL4A1.
(g) C6orf145, AGXT2L1, ADRB2, PNPLA4, SERPINE1, ZFP36L1, SKIL, JAG1, CCDC93, BMPR2, ZFP36L1, FAT4, JUN, ALOX5AP, FZR1, NR2F2, FHL3, COL4A1, CEBPD, and RAI2; or
(h) CTGF, GADD45B, BHLHB2, SERPINE1, NP, ZNF395, MLXIP, DNAJC7, AGXT2L1, RBMS1, TNFRSF12A, CCDC93, VEGFA, NR2F2, COL4A1, FNDC3B, SPSB1, PNPLA4, PAIP2B, and BMPR2.

20-26. (canceled)

27. The kit of claim 17 further comprising a gene chip with lung metastasis signature (LMS) gene markers.

28. The kit of claim 27 further comprising an estrogen receptor detector.

29. The kit of claim 17 further comprising an estrogen receptor detector.

30-31. (canceled)

32. The kit of claim 17 wherein the group of genes consists of twenty genes.

33. A method of treating cancer cells with high metastatic potential comprising;
obtaining a diagnostic signature from a cancer cells indicative of the metastatic potential of the cancer cells;
wherein said diagnostic signature is obtained by measuring levels in cancer cells from the patient of five or more markers selected from the group of genes typifying the TGFβ response in human epithelial cells;
comparing said diagnostic signature to a control signature; and
based on the comparison, providing anti-TGFβ therapy if the diagnostic signature is greater than the control signature by a threshold amount.

34. The method of claim 33 wherein the group of genes typifying the TGFβ response in human epithelial cells from which the five or more markers are selected consists of ABTB2, ACSBG1, ADCK2, ADRB2, AGTR2, AGXT2L1, AHI1, ALOX5AP, AMIGO2, ANGEL2, ANGPTL4, ANK1, ARFGAP1, ARHGAP12, ARID5B, ARL6IP2, ATF7IP, AVPI1, BET1L, BHLHB2, BHMT, BMPR2, BRDT, C13 orf15, C18orf25, C3orf28, C3orf52, C6orf145, C6orf148, CCDC93, CD163, CD1E, CD28, CDKN1A, CDKN2AIP, CEBPD, CENPF, CITED2, CITED2, CKMT2, COL1A1, COL4A2, COL8A2, CTGF, CUBN, CYBB, DDIT4, DNAJC7, DOPEY1, EDN1, ELK3, ETS2, FAT4, FGB, FHL3, FILIP1L, FLJ10357, FLT4, FLVCR2, FNDC3B, FSTL3, FZR1, GADD45B, GRB10, HLX, HMOX1, HNMT, HRH1, ID1, IL11, IL5, IRS1, JAG1, JMJD3, JUN, JUNB, LARP6, LBH, LEMD3, LMCD1, MAP3K4, MAS1, MGC14376, MLXIP, MTMR1, MYBL1, MYC, MYH11, NA, NCOR2, NDST1, NEDD9, NP, NPAS1, NR2F2, OLIG2, PAIP2B, PASK, PCTK2, PDGFA, PDLIM4, PFKFB3, PHLDB1, PKIA, PLK3, PNPLA4, PPP1R13L, PSCD1, PSCD1, PTH, PVRIG, RAB11FIP4, RAI2, RARA, RASL10A, RBMS1, RHOB, RNASE4, SERPINE1, SERTAD2, SGK, SKIL, SLC16A3, SLC17A3, SMAD7, SMOX, SMTN, SMURF1, SNAI1, SPHK1, SPP1, SPSB1, SSBP3, SYCP1, TBC1D2B, TBL1Y, TBPL1, TFEB, THPO, TMEPAI, TNFAIP8, TNFRSF12A, TPM1, TUBB4, TUFT1, UTP14A, VEGFA, YIPF5, ZEB1, ZFP36L1, ZNF318, ZNF395, and ZNF44.

35. The method of claim 33 wherein the group of genes wherein the group of genes typifying the TGFβ response in human epithelial cells from which the five or more markers are selected consists of:
(a) HMOX1, VEGFA, SERPINE1, FHL3, FNDC3B, COL4A1, YIPF5, ANGPTL4, TNFRSF12A, CTGF, JUN, ETS2, CCDC93, COL4A2, SMURF1, SPSB1, SMTN, JAG1, MLXIP, NEDD9, PAIP2B, RAI2, GADD45B, NP, AGXT2L1, ALOX5AP, RBMS1, C6orf145, AHI1, TPM1, SKIL, BHLHB2, SMOX, JUNB, SSBP3, ELK3, SNAI1, DNAJC7, NA, ADRB2, PNPLA4, FZR1, ZFP36L1, ANGEL2, BMPR2, NR2F2, PSCD1, IRS1, CEBPD, and FAT4,
(b) BHLHB2, COL4A1, CCDC93, JAG1, JUN, NR2F2, RAI2, RBMS1, ZFP36L1, AGXT2L1, ALOX5AP, C6orf145, FAT4, FHL3, GADD45B, HMOX1, SERPINE1, SMTN, SMURF1, SPSB1, TNFRSF12A, ADRB2, ANGPTL4, BMPR2, CTGF, ETS2, FNDC3B, FZR1, IRS1, NEDD9, PAIP2B, PNPLA4, SKIL, SSBP3, TPM1, VEGFA, ZNF395, AHI1, CEBPD, COL4A2, DNAJC7, JUNB, MLXIP, NP, PSCD1, SMOX, and YIPF5;
(c) HMOX1, VEGFA, SERPINE1, FHL3, FNDC3B, COL4A1, YIPF5, ANGPTL4, TNFRSF12A, CTGF, BHLHB2, JUN, ETS2, CCDC93, COL4A2, SMURF1, SPSB1, RBMS1, SMTN, and JAG1;
(d) FHL3, GADD45B, TNFRSF12A, ETS2, JAG1, SMURF1, FAT4, NR2F2, TPM1, JUN, CCDC93, HMOX1, RBMS1, BHLHB2, ZFP36L1, SSBP3, ZNF395, SKIL, FZR1, and RAI2;
(e), AHI1, C6orf145, IRS1, COL4A1, JAG1, SSBP3, ZFP36L1, ADRB2, RAI2, AGXT2L1, SPSB1, ALOX5AP, GADD45B, SMURF1, JUNB, SMTN, and NEDD9;
(f) IRS1, PSCD1, TPM1, C6orf145, NEDD9, HMOX1, RAI2, NR2F2, FAT4, SMTN, ZFP36L1, ALOX5AP, RBMS1, SMOX, ANGPTL4, PAIP2B, BHLHB2, RBMS1, JUN, and COL4A1.
(g) C6orf145, AGXT2L1, ADRB2, PNPLA4, SERPINE1, ZFP36L1, SKIL, JAG1, CCDC93, BMPR2, ZFP36L1, FAT4, JUN, ALOX5AP, FZR1, NR2F2, FHL3, COL4A1, CEBPD, and RAI2; or
(h), CTGF, GADD45B, BHLHB2, SERPINE1, NP, ZNF395, MLXIP, DNAJC7, AGXT2L1, RBMS1, TNFRSF12A, CCDC93, VEGFA, NR2F2, COL4A1, FNDC3B, SPSB1, PNPLA4, PAIP2B, and BMPR2.

36-42. (canceled)

43. The method of claim 33 comprising targeting genes with an expression level different than the control signature by a threshold amount.

44. The method of claim 33 wherein the cancer cells are breast cancer cells.

45. The method of claim 33 wherein the cancer cells are melanoma cells.

46. The method of claim 33 wherein the group of genes consists of twenty genes.
